SEQUENCE LISTING (58 sequences)

SEQ ID NO: 1 - DNA, 1143 bp, Aspergillus clavatus
  CDS (1)..(1143): Lys1
atgtcgagcaacaagatttggctgcgcgctgagaccaagcccgctgaagcgcggtctgccttgactcctaccacatgcaaggctcttatggatgccggctacgacgttactgtcgagcgttccacacaacgcatctttgatggtgaggataacatactctcctaccttatcggcgctcccctggttgaggaaggctcatgggtcaaggatgcccccaaggacgcttacgtcctgggtctcaaggagcttcccgaagatgacttccccctcgagcacgtccacatctctttcgcacactgctacaaggaacaaggtggctgggagaaggttcttagccggtggccccgtggaggcggcaccctcttggacttggagttcctcacagatgatgttggtcgcagggtagctgctttcggatactctgctggttatgcaggctctgctctggccgtcaagaactgggcctggcaattgacacaccccgagggcgagcctctgcccggcgagacaccctatgagaaccaagatcttctgatcgcgtccgtgaaggagtcgctggaggttggcaagaagcagtctggaaagtcgcccaaggtgcttgtcattggagctcttggccgttgcggcaagggtgctgtgcagctggccaaggatgttggcattcccgagtctgatatcatccagtgggatattgaagagaccaagaagggtggacccttccgtgagatcgttgaggacgttgatatcttcgtcaactgcatctacctctccgccaagatccctcctttcgtcaacgccgagaccctctctactcctaaccgccgcttgtctgtcatttgcgacgtgagcgctgacacaaccaacccccacaaccctatccccgtctactcgatcaccaccacctttgataagcccacggtgccagtgaccctgtccgccggcgcccagggtccccctctgagcgtgatcagcattgatcatctcccctctctcttgccccgtgagagctccgagatgttcagcgaagcgctgctgcctagcctgctgcagctcaaggacagaaagaacgctcgcgtctggaagcaggcggaggatctgtttaacgagaaggtcgctacgttgcccgagtcgatgcgcgcataa

SEQ ID NO: 2 - protein, 380 aa, Aspergillus clavatus
MSSNKIWLRAETKPAEARSALTPTTCKALMDAGYDVTVERSTQRIFDGEDNILSYLIGAPLVEEGSWVKDAPKDAYVLGLKELPEDDFPLEHVHISFAHCYKEQGGWEKVLSRWPRGGGTLLDLEFLTDDVGRRVAAFGYSAGYAGSALAVKNWAWQLTHPEGEPLPGETPYENQDLLIASVKESLEVGKKQSGKSPKVLVIGALGRCGKGAVQLAKDVGIPESDIIQWDIEETKKGGPFREIVEDVDIFVNCIYLSAKIPPFVNAETLSTPNRRLSVICDVSADTTNPHNPIPVYSITTTFDKPTVPVTLSAGAQGPPLSVISIDHLPSLLPRESSEMFSEALLPSLLQLKDRKNARVWKQAEDLFNEKVATLPESMRA

SEQ ID NO: 3 - DNA, 1125 bp, Aspergillus fumigatus
  CDS (1)..(1125): Lys1
atgtcaagcaataagatctggttgcgcgcggaaaccaagcctgccgaggctcggtctgctttgaccccgactacctgcaaggcccttatggatgctggctacgaggtgaccgtggaacgttccagacagcggattttcgacgttgtacagatcggcgcccccctcgtcgaggaaggttcatgggtaaaggacgcacccaaagatgcctacatcctcggtctgaaggagcttcccgaggacgattttccgcttgagcacgtacacatctcctttgcgcactgctacaagcaacaggctggctgggagaaggtgctcagccggtggccccgcggaggcggcaccctcttggacttggagttcctcacagatgagactggacgccgagtagctgcttttgggtactccgccggttacgcaggttctgctttggccattaagaactgggcctggcaattgacgcatcctgagggcgagccgcttcctggcgagactccctacgcaaaccaggatctgttgattgagtcagtgaaggagtcgttggagtctggcaagaagctgtccggcaggccgcccaaggtgcttgtcattggagctcttggacgctgtggcaaaggagcggttcagctggccaaggatgtcggcattcctgagtcggatatcatccagtgggatatagaagagaccaagaagggtggccccttcagagagattgtcgaggatgcagacattttcatcaactgcatctacctctccgctaagatccctcctttcgtcaacaccgaaactctgtcttctcctaaccgccgcttgtccgtcatttgtgacgtgagcgccgacacaaccaatcccaacaatcctatccctgtctattccatcacaaccactttcgacaagcccacagtcactgttcctcttccggaactggcccagggccctccattgagcgtgatcagcatcgaccacctcccctccctccttcctcgtgaaagctccgagatgttcagcgaagccttactgccgagcctactgcaactcaaggatagaaagaacgctcgtgtctggaagcaagcagaggacttgttcaacgaaaaggttgctaccttgcccgagtcgatgcgcgcttaa

SEQ ID NO: 4 - protein, 374 aa, Aspergillus fumigatus
MSSNKIWLRAETKPAEARSALTPTTCKALMDAGYEVTVERSRQRIFDVVQIGAPLVEEGSWVKDAPKDAYILGLKELPEDDFPLEHVHISFAHCYKQQAGWEKVLSRWPRGGGTLLDLEFLTDETGRRVAAFGYSAGYAGSALAIKNWAWQLTHPEGEPLPGETPYANQDLLIESVKESLESGKKLSGRPPKVLVIGALGRCGKGAVQLAKDVGIPESDIIQWDIEETKKGGPFREIVEDADIFINCIYLSAKIPPFVNTETLSSPNRRLSVICDVSADTTNPNNPIPVYSITTTFDKPTVTVPLPELAQGPPLSVISIDHLPSLLPRESSEMFSEALLPSLLQLKDRKNARVWKQAEDLFNEKVATLPESMRA

SEQ ID NO: 5 - DNA, 1119 bp, Botrytis cinerea
  CDS (1)..(1119): Lys1
atgtctggaactactcttcacctacgttctgagttgggcaaagctcttgagcatcgatccgctcttacccccaccaccgccaaagctctcatcgatgctggatacacaatcaacgttgagcgcagtccagagcgtatattcgacgatgaagagttcgagaaggttggtgctactcttgtgccagagaacacatggagacaagcaccaaaggatcacattatcattgggttgaaggaactgcccgtcgaagaatttcctctcgagcatgttcacgtacaattcgcacattgttataaacaacagggcggctgggacactgttctatcacgattccctcgtggaggtggaactctcttagatcttgagtttttgacagacgacagaggcagaagagttgcagcctttggataccatgctggatttgctggtgcagcattggcactcgaaaattgggcgtggcaactcacccacccagcatccgagcccttcccaagtgtatcgagctaccccaacgaagatgaattgattgtggatgttaagaaggcaatcgcagctggacaagagaagaccggcaaggcaccacgagttttggttattggtgcattaggcagatgtggaagtggagcagttgacctctgcttgagagctggtgtgccaaccgaaaatgtgttgaagtgggatatggccgagaccgctaagggagggccattcccagagatcgttgagagtgacatcttcatcaactgcatatatctcatgtccaagattccaaactttgtcgacatgcaaagcctcgataccccaaaccgcaaattgtcagtcgtctgcgatgtcagtgctgataccaccaaccccaacaacccaattccaatctatactgttgcaaccacattttcagagccaactgttccagttgaggtcaagggagaaccaagattaagtgtcatcagtattgatcacttgccaagtttattgccaagagaggcaagtgaggcatttagcaaggacttattaccaagtttgttgtctttgaaggattggagaaatactccagtctgggccaaggcagagaagttattccaggaaaaggttgctactttgcccaagaacgaggcatga

SEQ ID NO: 6 - protein, 372 aa, Botrytis cinerea
MSGTTLHLRSELGKALEHRSALTPTTAKALIDAGYTINVERSPERIFDDEEFEKVGATLVPENTWRQAPKDHIIIGLKELPVEEFPLEHVHVQFAHCYKQQGGWDTVLSRFPRGGGTLLDLEFLTDDRGRRVAAFGYHAGFAGAALALENWAWQLTHPASEPFPSVSSYPNEDELIVDVKKAIAAGQEKTGKAPRVLVIGALGRCGSGAVDLCLRAGVPTENVLKWDMAETAKGGPFPEIVESDIFINCIYLMSKIPNFVDMQSLDTPNRKLSVVCDVSADTTNPNNPIPIYTVATTFSEPTVPVEVKGEPRLSVISIDHLPSLLPREASEAFSKDLLPSLLSLKDWRNTPVWAKAEKLFQEKVATLPKNEA

SEQ ID NO: 7 - DNA, 1173 bp, Fusarium graminearum
  CDS (1)..(1173): Lys1
atgtctcaatacccccacatcctcctgcgcgctgaggagaagcccctcgagcaccgatctttctctccctcaattatcaagactcttgtcgacgctggatatcccgtctctgtcgagcgatcatctacagaccccaagttcaagcgcatctttgaagactccgagtatgaggctgctggtgcgcgcctcgtcaacgagggcacctggcccaacgccgaagccggaactttgattctgggcctcaaggagattcccgaggaagactttcctctcaagaacgaccacatttcatttgcccattgttacaagaaccaaggtggatgggaaaaggtcctcggccgcttccctcaaggaagcagcgttctatacgatttggagttccttgtcgatgagcaaggacgccgagtttctgctttcggtttccacgctggattcgctggcgctgccctcggtatcaagactcttgcccaccagcttcaggacccctcctccaagctcccctcagtggagacattcaccgatggtcgtggatactacctaaacgaggaggagctcgttaaccagatccgagaagaccttgccaaggccgagaagtctcttggacgcaagcccactgctctcgtcctcggtgctcttggacgatgtggtaagggtgccgttgacctgttcctcaaggctggcatgcccgacgagaacatcacccgctgggacttgaacgagaccaaggaccgagatggtccttatgaggagattgcccaggccgatgtcttccttaacgccatctacctttccaagcccatccctcctttcatcaacgaggagcttctcgccaagtctggccgcaacttggctgttgttatcgatgtctcctgcgacaccaccaaccctcacaaccccatccccatctactctatcaacaccacattcgaggagcccaccgtccccgtcgagatcaagaacgaccagaactctctccctctatcagttattagcatcgaccatctcccctcaatgctgccccgtgaggctagtgaggcctttagcgagggcctcaaggagtctctccttacactcaaggaccgcaagacttcgcgagtgtgggccgacgccgagaagctcttcaacgagaaggttgctctgcttcccgagtccctgcgaaccaagagagtttaa

SEQ ID NO: 8 - protein, 390 aa, Fusarium graminearum
MSQYPHILLRAEEKPLEHRSFSPSIIKTLVDAGYPVSVERSSTDPKFKRIFEDSEYEAAGARLVNEGTWPNAEAGTLILGLKEIPEEDFPLKNDHISFAHCYKNQGGWEKVLGRFPQGSSVLYDLEFLVDEQGRRVSAFGFHAGFAGAALGIKTLAHQLQDPSSKLPSVETFTDGRGYYLNEEELVNQIREDLAKAEKSLGRKPTALVLGALGRCGKGAVDLFLKAGMPDENITRWDLNETKDRDGPYEEIAQADVFLNAIYLSKPIPPFINEELLAKSGRNLAVVIDVSCDTTNPHNPIPIYSINTTFEEPTVPVEIKNDQNSLPLSVISIDHLPSMLPREASEAFSEGLKESLLTLKDRKTSRVWADAEKLFNEKVALLPESLRTKRV

SEQ ID NO: 9 - DNA, 591 bp, Fusarium oxysporum
  CDS (1)..(591): Lys1
atgtctgaatatccccacatcctacttcgcgctgaggagaagcctctcgagcaccgatctttctcccccgccgttatcaagacactcgtcgatgctggataccccatttccgtcgagcgatcgtccacagaccccaagtttaagcgtatctttgaggactcagagtatgaggctgctggcgctcgtcttgtcgatacgggtgtctggcccaacgctgagcctgggacaatcatcctggggctgaaggagatacccgaggaggacttccccctcaagaatgaccacatcacattcgctcactgctacaagaatcaagggggctgggagaaggtactcggtcgctgggcacaaggcggctcagttttgtatgacttggagttcttgcacgattctgaaggtcgtcgggtttcggccttcggtttccacgctggttttgcaggagctgcgctcggaattaagacgcttgctcaccagctgcaggactcgtcttctaagctcccttcagtcgagacattcaccgatggccgcggatactacctgaacgaggatgagctcgtcaacccagattcgcgaggatctcaccaaggctga

SEQ ID NO: 10 - protein, 196 aa, Fusarium oxysporum
MSEYPHILLRAEEKPLEHRSFSPAVIKTLVDAGYPISVERSSTDPKFKRIFEDSEYEAAGARLVDTGVWPNAEPGTIILGLKEIPEEDFPLKNDHITFAHCYKNQGGWEKVLGRWAQGGSVLYDLEFLHDSEGRRVSAFGFHAGFAGAALGIKTLAHQLQDSSSKLPSVETFTDGRGYYLNEDELVNPDSRGSHQG

SEQ ID NO: 11 - DNA, 1104 bp, Fusarium verticillioides
  CDS (1)..(1104): Lys1
atgtcgcctgtcgtcattcatctccggaccgagacaaagcctctagagcgtcgctctccattgtcacctgcgactgctaaggctttgctcggcgctggctacacagttcgagttgaggaatccccggaccgcatttacaagattgacgagttcagagatgttggggctgaaatcgtacccgctggctcgtgggtgaatgcacccaaggaggatattatcctgggattgaaggagattgaggcgaatggtacaccgttacttcacacctacattcactttgctcatgtattcaagaagcaaagcggctgggctactgagctgtcccgcttcgcaaacgcgggtggcttactatatgacttggaatttcttacggaccaagatggacgccgggtagctgcattcggatattgggcgggatatgccgggaccgctctggcactcctgtcttgggctcaccagctgctcaaccctggcgtaccccaaggaccggttcccgtcttcgactctgcctctgctttgactgaacttgtcaagggtaaagtcgacgctgcacgctccgccaaccacggcgcgctccctcgactgatcgtaatcggtgccctgggccgctgcggcaaaggtgccatcgcagcagctgaggccattggtgttagtgacattctgaaatgggacattgctgagacgagcaagggtggcccatttcctgaagtcgcctcgtctgatatttttgtgaactgcgtctacctaggttccaacaagattccacccttcacaacttttgaagcactctcaggacccggcagacgactccgggtcatttgcgacgtcagttgcgaccccaacagcgagaataaccctgttcccgtctactccagctacagttcgttcgaaaacccgactgtccctgcttctgagcatattgatggtcctgaactgcgcatcattgctatcgatcatcttcctaccatggttgcacgcgagtcgagtgacgagtattcctcactacttctgccaagtttgttgactctggaccgccgggatactgagggggtttggcagcgagcagaacggatctttcgcgagaaggttgcagagctgccctag

SEQ ID NO: 12 - protein, 367 aa, Fusarium verticillioides
MSPVVIHLRTETKPLERRSPLSPATAKALLGAGYTVRVEESPDRIYKIDEFRDVGAEIVPAGSWVNAPKEDIILGLKEIEANGTPLLHTYIHFAHVFKKQSGWATELSRFANAGGLLYDLEFLTDQDGRRVAAFGYWAGYAGTALALLSWAHQLLNPGVPQGPVPVFDSASALTELVKGKVDAARSANHGALPRLIVIGALGRCGKGAIAAAEAIGVSDILKWDIAETSKGGPFPEVASSDIFVNCVYLGSNKIPPFTTFEALSGPGRRLRVICDVSCDPNSENNPVPVYSSYSSFENPTVPASEHIDGPELRIIAIDHLPTMVARESSDEYSSLLLPSLLTLDRRDTEGVWQRAERIFREKVAELP

SEQ ID NO: 13 - DNA, 1173 bp, Fusarium verticillioides
  CDS (1)..(1173): Lys1
atgtctgaatatcctcacatccttcttcgcgccgaggagaagcctctcgagcaccgatctttctcccccgcggttatcaagacactcgtcgatgctggataccccatctccgtcgagcggtcatccaccgatcccaaatttaggcgtatctttgaggactctgaatatgaagctgctggtgctcgtcttgtcgacacgggtgtctggcccaacgctgagcctgggacaatcatactcggattgaaggagattcccgaggaggatttccctctgaagaatgatcacatcacatttgcacactgctacaaaaaccagggaggctgggagaaggttctcggtcgttggtctcgcggcgggtctactctgtatgacttggagtttttgcatgacgctgaaggccgacgtgtttctgcgtttggtttccacgcaggcttcgccggggctgcgcttggaataaagacactttcccaccagctgcaggacccgtcatctaagcttccgtctgtcgagacattcaccgatggccgcggatattacctgaacgaagatgagctcgtcaaccagattcgcgaggatctcgccaaggctgagaaggctctcggacgcaagcccactgctctcgtccttggtgctcttggacgatgtggcaagggtgctgtggaccttttcctcaaggccggcatgcctgatgagaacatcacccgttgggacttgaacgaaaccaaggaccgtgatggaccttacgaggaaattgcgaaggctgatgtcttccttaacgccatctacctctccaagcccatcccccctttcatcaaccaagaactcctcgccaagaagggtcgcaacctcgctgtagtcatcgacgtttcttgtgataccacaaacccccacaaccccatccccatctactccatcaacaccaccttcgaggaccccaccgtccccgtcgagatcaaggacgaccagaacaacctccccctttccgtcatcagcatcgaccaccttccctccatgcttccccgcgaggctagtgaggcctttagtgagggtctcaaggagtctctgcttacactcaaggatcgcaaaacttcgcgggtgtgggctgatgctgagaagctcttccatgagaaggttgctacgttgccggaggagttgagaaccaagaacgtttaa

SEQ ID NO: 14 - protein, 390 aa, Fusarium verticillioides
MSEYPHILLRAEEKPLEHRSFSPAVIKTLVDAGYPISVERSSTDPKFRRIFEDSEYEAAGARLVDTGVWPNAEPGTIILGLKEIPEEDFPLKNDHITFAHCYKNQGGWEKVLGRWSRGGSTLYDLEFLHDAEGRRVSAFGFHAGFAGAALGIKTLSHQLQDPSSKLPSVETFTDGRGYYLNEDELVNQIREDLAKAEKALGRKPTALVLGALGRCGKGAVDLFLKAGMPDENITRWDLNETKDRDGPYEEIAKADVFLNAIYLSKPIPPFINQELLAKKGRNLAVVIDVSCDTTNPHNPIPIYSINTTFEDPTVPVEIKDDQNNLPLSVISIDHLPSMLPREASEAFSEGLKESLLTLKDRKTSRVWADAEKLFHEKVATLPEELRTKNV

SEQ ID NO: 15 - DNA, 1223 bp, Mycosphaerella fijiensis
  CDS (3)..(1223): Lys1
gaaggtcaacacttaaaaaagactcatattccgcgtcgactataatcgtacaagatcagtgtctcacaatgtcgcctacagtccttcatttcagagcagagaccaagccattagagcatcgctcggcagtcacaccgacaatcgcaaagaaacttgtcgaagctggctatgaagtccacgtcgaacgatcgcagctcagcatattcgcagactccgagtatgaaggcactggtgctaagctagtgcctactggatcgtggacagaggcaccgaaggagcacattgtcattggcttgaaggaactccccgaggaggactttcctttgaagcatgttcatgtgcaatttgctcactgctacaaaggacaaggtggctgggacaaggttctcagtagatttccaaacggcagaggcacattacttgaccttgaattcttggaagatgagcaaggacgaagagtcgctgcatttggctaccatgccggatttgctggtgccgcacttgctctcatggcctggtctcaccagctcgtgcacggcaaggacagccctcttccgggagtgacaccatacgaaaacgaaggtctccttatttccgatgtcaagaaggccatcgaagctggcaaggccaagggcggttgcctcccaagagtgctcgttattggcggactcggcagatgcggtcgtggtgccgttgacctctgtgtcaaggctggcgtggaagacattctaaagtgggatcttccagaaacaagtgccaaaccaggaccataccaggagatcatcgagtctgacgtgtttgtcaactgcatctacttgtcggccaagatcccgcccttcatcgaccaggcatctctcgcatcgccaaaccgcaaactgtctgtggtctgtgatgtatcctgcgataccacgaacccaaacaatcctatcccaatctacgacatcaacacgactttcgacaagccaacggtgcctgtcaagctctccgctgaggccaatgatcttccgttgagcgtaatcagtatcgaccatctcccatccttactgccgcgagaagcttccgaagctttcagctccgcgctgctgcccagcttgcttcaattgaacgactggaagaacgcacgtgtttggcaacaagccgagaagctgttcaaggacaaatgcgccactctcccagaaggcgcaatcgatagccatgccgagcttcttgcttcgcaatcttga

SEQ ID NO: 16 - protein, 406 aa, Mycosphaerella fijiensis
RSTLKKDSYSASTIIVQDQCLTMSPTVLHFRAETKPLEHRSAVTPTIAKKLVEAGYEVHVERSQLSIFADSEYEGTGAKLVPTGSWTEAPKEHIVIGLKELPEEDFPLKHVHVQFAHCYKGQGGWDKVLSRFPNGRGTLLDLEFLEDEQGRRVAAFGYHAGFAGAALALMAWSHQLVHGKDSPLPGVTPYENEGLLISDVKKAIEAGKAKGGCLPRVLVIGGLGRCGRGAVDLCVKAGVEDILKWDLPETSAKPGPYQEIIESDVFVNCIYLSAKIPPFIDQASLASPNRKLSVVCDVSCDTTNPNNPIPIYDINTTFDKPTVPVKLSAEANDLPLSVISIDHLPSLLPREASEAFSSALLPSLLQLNDWKNARVWQQAEKLFKDKCATLPEGAIDSHAELLASQS

SEQ ID NO: 17 - DNA, 1098 bp, Magnaporthe grisea
  CDS (1)..(1098): Lys1
atgtcgtccattctgcacttgcggtcagagaccaagccattggagcaccggtccgccctaactcccactaccaccaaggccttgattgaggccggttacaccgtcaatgtcgagcgcagtcccgtccgcatcttcgacgacgccgagttcgaggctgttggcgcaaccctcgtccccgagggcagctgggagcagattcccaaggaccacatcgtcattggactgaaggagttggaggagaaggacttccctttgaagcacacacacatccagttcgcccactgctacaagaaccaaggaggctgggacaaggtcctccggaggtatcacgatggcgagggaatgctcctcgacattgagttcctcgagaaggacggccgtcgtgttgctgctttcggctactgggccggctttgccggtgccgcgctggcccttcagaactgggcctggcagctgaccaacagcggcgagcccctgccgagcgtcgagagccgccctaacgaggctgccttggtcggcgacatcaaggaggctcttgctgggggcaaggaaaaggccggcaggctgccccgcgtcatcgtcatcggtgccctgggccgctgcggtcgtggtgccgtcgacatgtgcaagaaggccggcataccggatgagaacatcctcaagtgggatatggaggagacggcccccggtggaccgttcaaggagattgtcgagagtgacatctttgtcaactgcatctacctgaacaagcagatcccgccgtttgtgaccatggagtcgctcaacacgccagagcgcaagctctccgtcatcaacgactgctcggctgacacgaccaacccctttaaccctgttcccgtgtacactgtggccacgactttcgacaagcccactgtgccggtcgatggcttgagccatggcccgcccctgagcgttatcagcatcgaccacctgccgagtctgctcccgcgggaggcttcggagtcgttcagcaacgacttgttgccatatctcctgaagctgaaggactggaagagtgacccggtgtgggctggcgccgagaagctgttccacgagaaggtcaagaccctgccgtag

SEQ ID NO: 18 - protein, 365 aa, Magnaporthe grisea
MSSILHLRSETKPLEHRSALTPTTTKALIEAGYTVNVERSPVRIFDDAEFEAVGATLVPEGSWEQIPKDHIVIGLKELEEKDFPLKHTHIQFAHCYKNQGGWDKVLRRYHDGEGMLLDIEFLEKDGRRVAAFGYWAGFAGAALALQNWAWQLTNSGEPLPSVESRPNEAALVGDIKEALAGGKEKAGRLPRVIVIGALGRCGRGAVDMCKKAGIPDENILKWDMEETAPGGPFKEIVESDIFVNCIYLNKQIPPFVTMESLNTPERKLSVINDCSADTTNPFNPVPVYTVATTFDKPTVPVDGLSHGPPLSVISIDHLPSLLPREASESFSNDLLPYLLKLKDWKSDPVWAGAEKLFHEKVKTLP

SEQ ID NO: 19 - DNA, 453 bp, Moniliophthora perniciosa
  CDS (1)..(453): Lys1
atgaaattcacactccctcgtcttcgaccactcaccattggaatccgccgggaagaccctactcgaatttgggaacgacgggctcctctaacccccgactccgtttacgagcttgtcaaggacaaggcggtacaagttcatgtcgaaggctgcgacaggaggatattcaaggacgaggaatacataaaggccggtgcaacaatccgacccaacctgaacgatgcacatgttgtaatgggcattaaagagcccccattggacagattattgcttgatcctcttcctctatccaacaccacgtcgaaacatgagcgaacatacatgaagttttctcatacttggaaaggtcaagcctacaatatgccattactttctgcattcttaaacattcacccattccatggcgcttataatgacccactggcccacactaattggataatgaactcctga

SEQ ID NO: 20 - protein, 150 aa, Moniliophthora perniciosa
MKFTLPRLRPLTIGIRREDPTRIWERRAPLTPDSVYELVKDKAVQVHVEGCDRRIFKDEEYIKAGATIRPNLNDAHVVMGIKEPPLDRLLLDPLPLSNTTSKHERTYMKFSHTWKGQAYNMPLLSAFLNIHPFHGAYNDPLAHTNWIMNS

SEQ ID NO: 21 - DNA, 1116 bp, Puccinia graminis
  CDS (1)..(1116): Lys1
atgtcaggcgatcaaccactcctatggctgcgatgcgagaccaaaccgttcgagcatcgctcagcattaactcccttgaccgccaaaaaactcatcgatgctggattcaagctggtagtagaacgggatcctcaacggttcttcgctgatgacgaatttgcaaaagtcggatgtgaactggtcgaacacaactcgtggcccaaggctccctccaatgcaattatcatcggattgaaggagttgcctccgaatgatgattcgcccctcatccatacccatgtcatgttcggccattgttacaagcaacaagcaggctaccaggacatcctaagtcgattcaaacgaggcgggggaactttgctcgacatggaattcttgcaggacgagcacaccaaacgcagagtggctgcctttggcttccacgccggattcaatggctctgctgtcggtctacttgcgcttgggtcgatgttatccggggagggaagtttgaaaggattgaagcctttcaaagatgaagacgaattgattactcgagggaagaaagaattcgatcgagtggtcgccaagctaggtcgacatcctaaggccctagtgattggttcattgggacgctgtggctccggagctgttacatttttcaagaaaattggattgaacaaggatgacgtcgttgaatgggacatggcagagactgccaaaggtggcccattccaagaaattcttgaagccgatatcttcatcaattgcatctacttgagtagcaaaatccccagctttgtcacccgggagacaatagccgcagcaggagattctcgccagctgagggttgtagtcgatgtgtcctgtgataccaccaatccgaacaatcctattccgatatatgacgtcaacactacattcgattctccaaccgtgcctgtccagctagatgctggactgccaagcttagaagtttgctcgatagatcatctgccaacacttctgcccagagaagcctccgaacaattttcgaacgatctccttcctacgcttttgcaattaaaaacgttggatcaatctaaagtttggactgaagcaagggatttgtttcacaagatggtcaattccatctag

SEQ ID NO: 22 - protein, 371 aa, Puccinia graminis
MSGDQPLLWLRCETKPFEHRSALTPLTAKKLIDAGFKLVVERDPQRFFADDEFAKVGCELVEHNSWPKAPSNAIIIGLKELPPNDDSPLIHTHVMFGHCYKQQAGYQDILSRFKRGGGTLLDMEFLQDEHTKRRVAAFGFHAGFNGSAVGLLALGSMLSGEGSLKGLKPFKDEDELITRGKKEFDRVVAKLGRHPKALVIGSLGRCGSGAVTFFKKIGLNKDDVVEWDMAETAKGGPFQEILEADIFINCIYLSSKIPSFVTRETIAAAGDSRQLRVVVDVSCDTTNPNNPIPIYDVNTTFDSPTVPVQLDAGLPSLEVCSIDHLPTLLPREASEQFSNDLLPTLLQLKTLDQSKVWTEARDLFHKMVNSI

SEQ ID NO: 23 - DNA, 3045 bp, Phytophthora infestans
  CDS (1)..(3045): Lys1
atgtcgggcaagtgtgtaggcatcgtccgcgaggtatacaacaaatgggagcgtcgagcgccgctcacacccgcacacgttaaggagctagtggcgcgtggcgtgcaggtgctggttcagccgtccacggcacgcgtcttctcagacgaccagtatgttcgcgccggagccacactgtccgagaatttggcgccggcgaatgtcattgtgggggtcaagcaggtgcccgagccggcactgctggcggacaagacgtacctcttcttcagtcataccatcaaagcgcagcccgagaacatggcgctgctggacgccgtgctacagcgtcgcgtcacgctaatcgactacgaatgtatcacagaggagagtggtaagcgtctcattgccttcggcggaaatgccggtcgtgcaggaatgattgcgggcttccgaggtctcggagaacgtctcatcaacatgggaatctcctcgcccttcgtcaacatcgcgtcctcctacatgtacgcagacttagagcacgctaaagacgcagtggaagctgcaggtagaaggattcgaacagatggtctgccgggtgacttggcgccaatgacattcgcctttactggcaatggcaatgtgtctaaaggagctcaggagatcttcaagctcatgccgcacgagatggtgcatccttcggaactaccaaagctgccaaaaaacaatcgtattctctacggtacagtcatcgacgatccggcctactttgttaagcctcagccgggtttcacgggtgagactacgagagctcactactaccagaatccacaccaatacgaagctgctttccatgaaaaagtgttgccatacacctcaatgctcgtcaactgtatgtactgggacgaccgttttccaagattggccacgcgagagcagattcgtgagcttcgcaagtctgggaaccacaaattgctcgggatcgctgatatctcgtgcgatatcggcggaagtatcgagtttctggagcgtgctactgaaattgagcgtccgttcgctctgtacgacgttgttgaagataaaatgcgcgaagatggagacagccgaggcctcgaaggagacgacggcattatgatgatgggtgtcgatatcttaccgagtgaactggcacgcgagtcgagtcagcagttcggtgaccgcttggtcggatacgtcacggcgctgtcgagtgcttcttcgtccaacgttcctctacatgaacagaaagagctgcctgctgagctacgtggagcttgtatcgccagcaatggtgtgcttgcacctaaatacgagtatatccaccgtatgcgagccgagcgcgagcgcagcaagcagtacaagttcttggatgctcaacaacaggtcgctgggagtacgtgcgttctactggaggggcacctgtttgataccggacttattaaccaggtactcaacctcattgaagaccatgatgggggtttccaccttctagactgcgaagtacgcccaaatgttggagtcggggacagtggtgacagcaccagttcgaacgccatcgtgcagattagtatgagtgaccgtgaagcgctggatgctatcatcaccaagatccgctccttagctgagcttacttctggtgcgaaggctacggtcacagaactacccgatctgtgcggcacagactactccaagacgcgaggtgtcgtgagaaaagatgcagcagccaatactatggctgaagtatcggtctccagcccgaagaagcggaaggttgtgtgttttggcgctggattggtggcgtcaccacttgtggagtatctatcaagagagcaaggaaacgaagttcatgtggtgtcgggtattgagagggaagtaaaggagatgatgcgcaaaatctctcgtcgaaacatcaagcctcacgtagtgaacgtcgctgaagatgctgctggagtcgacaaactttgcgctgaagccgattgcgttgtgtcgctactgccagcgaccatgcacacgacaattgctcagcgctgcattcagcatgcgactcctctcgtgacggctagctatgtgtcaccagaaatgaaggagctggacgctagggcaaagaaggcaggtattccgatcctctgtgagattggtctcgaccctggtatggatcacatgagtgccatgaaagtcattgatgaagtgaaggccctctccgggaaggtcatgaccttctcgtctgtctgtggcggcttgccagctcctgaagcagcggacaacgccattggttacaagttcagttggagtccgcgtggtgtgctcacggcggcactgaatgcagcccagtaccgcaaagacggcaaagtcatcaacgtggcaggtgaagacttactcaatagaagcgaacgcgtaaacttcttgccggcgttcaacatcgagcagatcccgaaccgtaattcgcttccttacggcgatatctatggcatcccagaggcacactcgctgtatcgtggaactctgcgatacggtggatgctgtcaaattctgtaccagctacgcaagcttggcctctttgacatggacccatccaaacccattccagcgacttggccggatcttctgacgcagttgggtggtttccaaggtcttcgcgaagacgcccatgggttccttcagtggcttggtgcctttgataaatcaaatcctgtggttaaagccccgtccatccttgacgctttctgcgctctcctgcaagataagttgtcgtaccaaccaggagaacgcgacatggccatcatgcatcatgagttcggcatcgagtacgaggatggcaagaaggaaaagcgtacgtcaacattcgtgggctacgggtccgagaagggcgacacgatcatggctaaaacggttggattaagtgctgctatcggcgtccagttgatcttgcaggacgcagttcaaggtcgtggtgtgcttacccccacaacccccgacatctacggtcctgcactggcccgtctcgaggtcgaaggtgtgcgcttcattgaaaaaacattcccacagcattaa

SEQ ID NO: 24 - protein, 1014 aa, Phytophthora infestans
MSGKCVGIVREVYNKWERRAPLTPAHVKELVARGVQVLVQPSTARVFSDDQYVRAGATLSENLAPANVIVGVKQVPEPALLADKTYLFFSHTIKAQPENMALLDAVLQRRVTLIDYECITEESGKRLIAFGGNAGRAGMIAGFRGLGERLINMGISSPFVNIASSYMYADLEHAKDAVEAAGRRIRTDGLPGDLAPMTFAFTGNGNVSKGAQEIFKLMPHEMVHPSELPKLPKNNRILYGTVIDDPAYFVKPQPGFTGETTRAHYYQNPHQYEAAFHEKVLPYTSMLVNCMYWDDRFPRLATREQIRELRKSGNHKLLGIADISCDIGGSIEFLERATEIERPFALYDVVEDKMREDGDSRGLEGDDGIMMMGVDILPSELARESSQQFGDRLVGYVTALSSASSSNVPLHEQKELPAELRGACIASNGVLAPKYEYIHRMRAERERSKQYKFLDAQQQVAGSTCVLLEGHLFDTGLINQVLNLIEDHDGGFHLLDCEVRPNVGVGDSGDSTSSNAIVQISMSDREALDAIITKIRSLAELTSGAKATVTELPDLCGTDYSKTRGVVRKDAAANTMAEVSVSSPKKRKVVCFGAGLVASPLVEYLSREQGNEVHVVSGIEREVKEMMRKISRRNIKPHVVNVAEDAAGVDKLCAEADCVVSLLPATMHTTIAQRCIQHATPLVTASYVSPEMKELDARAKKAGIPILCEIGLDPGMDHMSAMKVIDEVKALSGKVMTFSSVCGGLPAPEAADNAIGYKFSWSPRGVLTAALNAAQYRKDGKVINVAGEDLLNRSERVNFLPAFNIEQIPNRNSLPYGDIYGIPEAHSLYRGTLRYGGCCQILYQLRKLGLFDMDPSKPIPATWPDLLTQLGGFQGLREDAHGFLQWLGAFDKSNPVVKAPSILDAFCALLQDKLSYQPGERDMAIMHHEFGIEYEDGKKEKRTSTFVGYGSEKGDTIMAKTVGLSAAIGVQLILQDAVQGRGVLTPTTPDIYGPALARLEVEGVRFIEKTFPQH

SEQ ID NO: 25 - DNA, 3015 bp, Phytophthora ramorum
  CDS (1)..(3015): Lys1
gtgggcatcgtgcgcgaggtgtacaacaagtgggagcgccgcgcgccgctcacccccgcgcacgtccgcgagctcgtgcagcgcggcgtgcaggtgttggtgcagccgtctacggcgcgcgtcttctcagacgagcagtacgtgcgagcgggcgccaagctggccgaggacctggcgcccgccaatatcatcgtgggcgtcaagcaggtaccggagccggcgcttctggcggacaagacatacctcttcttcagccataccatcaaggcgcagcctgagaacatggcgctcctggatgccgtgctgcagcgccgcgtcacgctcatcgactacgagtgcatcacagaggagagcggcaagcgcctcatcgccttcggaggcaacgcggggcgagcaggaatgatcgcgggcttccgggggcttggggaacgcctcatcaacatgggcatctcctcgccctttgtgaacgtagcgtcggcctacatgtactcggacttggagcacgcaaaagacgccgtagaagctgcgggcaggaggattcggtccgatggattgccaggcgagctggcgcccatggccttcgccttcacgggcaatggcaacgtgtccaaaggggcgcaggagatcttcaagctcatgccacatgaaatggtgcatccgtcagagctgccaaacttgccgaagaacaaccacatcctgtacggcaccgtggttgatgacccggccttctttgtgaagccgcaggctggcaacagcggcacggcctcgcgcgcacactactaccagaacccacaccagtacgaccctgccttccacgagaaggtgctgccgtacacgtccatgttggtgaactgcatgtactgggacgaccgtttcccgcgattggttacgcgcgaacagatccgtgagcttcgaggctctggaaaccaaaagctgctgggcattgccgatatttcgtgcgatattggcggcagtgttgaatttttggagcgcgcgacggaaattgaacgcccgttcgcgctgtacgatgtcgctgaagacaaaatgcgcgaggatggcgatagtcgagggctcgaaggggataacggcatcatgatgatgggggtcgatatattgccaagcgaacttccacgcgagtctagtcaacagttcggcgaccgtctggtcgcatacgtggcggcgctgtcgaatgcttcctcatccagtgtcccacttcatgagcaaaaggagctacctgctgagctccgtggcgcctgcatcgcaagcaaaggagtactcgccccaaaatacgagtacattcaccgtatgcgtgccgaacgtgagcgcagtaagcagtacaagttcctggatgcgcagcaggaagttgcggggagcacgtgtattctgcttgaaggtcacctcttcgatacaggacttatcaaccaagtgctcaacctcattgaggaccatgatggcggattccaccttgtggactgtgaagtgcggccgaacgttgctgtcagcgacagtggcgactgtaccatatcgaatgctatcgtccagataagcatgaacgatcgagctgcgctggatgaaattatctcaaaggttcgctctcttgctgaccttacctccggtgccaaggctactgtcactgagcttcccgacttgtgcggtacaaactactcgaagacacgtggtgctgtagtacggaaggacgcggctgccaatacgactaccgacgtgtcggtctcaagcccaaagaggcgaaagattgtatgctttggcgccgggttggtggcatcgccccttgtggagtatctgtcgcgtgaacaagtcaacgaagttcatgtggtgtctggcctcgagggtgaggtaaagggaattatgcgcaagatctctcgccgaaacatcaagcctcatgttgtaaacgtagctgaagacactgccggagttgacaagctttgtgcagaagctgattgtgtcgtgtcacttttgccagcgaccatgcacacaacgattgccgagcgctgtattcagcacgcgacaccacttgtgactgcaagttatgtgtctccggagatgaaagagctggactccaaggcaaagcaagctggcatccctatcctctgtgaaattggtcttgaccctggcatggatcacatgagcgcgatgaaagttattgatgaggtgaaggcgcactctgggaagatcaagtctttctcgtctgtatgtggcgggctgccggcgcctgaggcagcggacaacgctattggttacaagttcagctggagtccgcgcggtgtgctaacagcagcgctgaatgcagctcaataccgcaaagacgacaaagtcattaatgtggccggtgaagacttgctaaacagcagtgagcgcgtgaacttcctgccagcgttcaacattgagcagattccaaaccgtaattcgctgccgtacggcgatatctacggcattccggaagcacactcattgtatcgcggcactctgcgctatggtggctgctgtcaaattttgtaccaactacgtaagctcggcctcttcgacatggacccatccaagcccattccagctacttggccagaccttctcactcaattaggtggacaccaagaccttcgtgaagatgccagcgggttccttcagtggcttggtgcctttgatcgcaccacgcctattgttaaggcaccgtccgttctcgacgctttctgtacattgctgcaggacaagttatcgtaccagccgggtgaacgcgacatggccatcatgcaccatgaattcggcattgagtacgaagatggcaaaagggaaaaacgcacgtcaacgttcgtgggctatggctccgagaagggcgacacaatcatggcaaaaactgtcggcttgagtgcagctattggcgtgcagctgattttgcaggacgccgtccagggccgaggtgtgctaaccccgacaacccctgacatctacggcccagccctcgcccgtctcgaagtcgaaggtgtgcgcttcatcgagaag

SEQ ID NO: 26 - protein, 1005 aa, Phytophthora ramorum
VGIVREVYNKWERRAPLTPAHVRELVQRGVQVLVQPSTARVFSDEQYVRAGAKLAEDLAPANIIVGVKQVPEPALLADKTYLFFSHTIKAQPENMALLDAVLQRRVTLIDYECITEESGKRLIAFGGNAGRAGMIAGFRGLGERLINMGISSPFVNVASAYMYSDLEHAKDAVEAAGRRIRSDGLPGELAPMAFAFTGNGNVSKGAQEIFKLMPHEMVHPSELPNLPKNNHILYGTVVDDPAFFVKPQAGNSGTASRAHYYQNPHQYDPAFHEKVLPYTSMLVNCMYWDDRFPRLVTREQIRELRGSGNQKLLGIADISCDIGGSVEFLERATEIERPFALYDVAEDKMREDGDSRGLEGDNGIMMMGVDILPSELPRESSQQFGDRLVAYVAALSNASSSSVPLHEQKELPAELRGACIASKGVLAPKYEYIHRMRAERERSKQYKFLDAQQEVAGSTCILLEGHLFDTGLINQVLNLIEDHDGGFHLVDCEVRPNVAVSDSGDCTISNAIVQISMNDRAALDEIISKVRSLADLTSGAKATVTELPDLCGTNYSKTRGAVVRKDAAANTTTDVSVSSPKRRKIVCFGAGLVASPLVEYLSREQVNEVHVVSGLEGEVKGIMRKISRRNIKPHVVNVAEDTAGVDKLCAEADCVVSLLPATMHTTIAERCIQHATPLVTASYVSPEMKELDSKAKQAGIPILCEIGLDPGMDHMSAMKVIDEVKAHSGKIKSFSSVCGGLPAPEAADNAIGYKFSWSPRGVLTAALNAAQYRKDDKVINVAGEDLLNSSERVNFLPAFNIEQIPNRNSLPYGDIYGIPEAHSLYRGTLRYGGCCQILYQLRKLGLFDMDPSKPIPATWPDLLTQLGGHQDLREDASGFLQWLGAFDRTTPIVKAPSVLDAFCTLLQDKLSYQPGERDMAIMHHEFGIEYEDGKREKRTSTFVGYGSEKGDTIMAKTVGLSAAIGVQLILQDAVQGRGVLTPTTPDIYGPALARLEVEGVRFIEK

SEQ ID NO: 27 - DNA, 3048 bp, Phytophthora sojae
  CDS (1)..(3048): Lys1
atgacgggcaagtgcgtgggcatcgtccgcgaggtgtacaacaagtgggagcggcgcgcgccgctcacgcccgcgcacgtccgggagctcgtgcagcgcggcatccaggtgctggtgcagccgtccacggcgcgcgtcttctcggacgagcagtacgtgcgcgcgggcgccactttgtccgaggacctggcgcccgccaacgtcatcgtgggcgtcaagcaggtgcccgagccggcgctgctggccgacaagacgtacctcttcttcagccacaccatcaaggcgcagccggagaacatggcgttgctggacgccgtgctgcagcgccgcatcacgctcgtcgactacgagtgcatcacagaggagagcggcaagcgcctcatcgccttcgggggcaacgccggccgcgccggtatgatcgcgggcttccgggggcttggagagcgcctcatcaacatgggcgtctcctcgcccttcgtgaacgtcgcgtccgcctacatgtactcggacttggagcacgccaaggacgcggtggaggccgcgggcaagaggatccgctcggacggactgccgagcgagctggtgcccatgacgttcgcattcacgggcaacggcaatgtgtcaaagggcgcgcaggagatcttcaagcttatgccccacgagatggtgcatccgtcggagctgcccaagctaccgaagaacaaccgtattctgtacggcacagtgatcgacaacccggactacttcgtcaagccccagccgggattcagcggcacaccctcccgcgcgcactactaccagaacccacaccagtacgagcctgctttccacgagaaggtgctgccctacacgtccatgctggtgaactgcatgtactgggacgaccgcttcccgcggctagtcacgcgagagcagatccgagagcttcgaggctctgggaaccataagctgctggggatcgcggacatttcgtgcgatattggcggcagcgtggagttcttggagcgcgccacggagatcgaacgaccattcgctctgtacgatgtcgctgaagacaagatgcgtgaggacggcgacagtcgaggccttgaaggcgacgatggcattatgatgatgggcgtcgatatcctgccgagcgagttggcgcgcgagtcaagccagcagtttggtgaccgcttggtgggatacgtcacggcgctatcgagtgtcacttcatcgaatgtacctctgcacgagcaaaaggagctgccggctgaactgcgtggcgcttgtattgccagcaaaggtgttttggcgcccaggtacgagtacattcatcggatgcgtgccgaacgtgagcgcagcaagcagttcaagttcctggatgctcagcaggaggttgcaggcagcacgtgcctgttactggaaggacacctctttgatacaggcctcatcaaccaggtactgaatctcatcgaggaccacgatggaggcttccacctcgtggactgcgaggtccgaccgaacattgcagccggagacagcggcttcagcactgtgtcgaatgctatagtccaggttagcatgagtgaccgtgctgctttggacgacattattgcgaagatccgctcccttgccgatcttacgtctggtgccaaggccatcgtcacggagcttcccgacttgtgcggcacgaattattcgaagactcgtggtgccgtggtgcgcaaggatgctgctgctaatacggcggcggacgtgtcagtctcgagcccgaagaagcgacagatcgtgtgttttggcgctggattagtggcatccccgcttgttgagtacctgtctcgcgagcaaggcaatgaagtgcacgtggtgtccggtatcgagggtgaagtgaagggggtgatgcgcaaaatctctcgccgtaacattaagcctcacgtggtgaacgtagctgaggacggtgctggcgttgacaagctctgtgcagaagccgattgtgttgtgtcactattaccagctacaatgcacacaaccatcgcccagcgttgcatccaacatggaacgcctctcgtgactgcaagctacgtgtctccggagatgaaagagttggatgccaaagccaagaaagccggcattccaatactgtgtgaaattggccttgatcctggcatggatcacatgagcgctatgaaggtcatcgacgaagtgaaggctcactccggaaagatcctgtcattttcatccgtgtgtggtggacttccagctcctgaagctgctgacaacgccattggttacaagttcagctggagtccgcgcggcgtgcttactgcagcactgaacgcggcgcaataccgcaaagacggcaagatcgtcaacgtcgcaggcgaagacttgcttaacagcagcgaacctgtgaacttcttgcctgcgtttaacatcgagcagatcccgaaccgtgactcactgccgtacggcgagatctacgtcattcctgaggcgcactcgctgtatcgtggaacactgcgctacggaggttgttgccgaatcctgtaccagctgcgcaagcttggtctctttgacatggacccgtccaagcccattccagctacgtggccagaccttatctcccagcttggtggacaccaaggccttcgcgaagacgccaatggattccttcaatggctcggtgccttcgatcacagcactcccgtagtgcgagccccatccatcctcgacgcattctgcgcactgttgcaggacaagttatcttaccaacctggagaacgtgatatggccatcatgcaccatgaattcggcatcgagtacgaagacggcaagaaggaaaagcgcacgtcaacgttcgtgggttacggctccgataagggcgacaccatcatggctaaaaccgttgggttgagtgcagcgattggtgtgcagttgatcttgcaggacgcagtccaaggccgaggcgtgcttacaccgacgacccctgacatctacgggccagccctcgcgcgacttgaggttgaaggtgttcgcttcatcgaaaagacgttcccgcagccttaa

SEQ ID NO: 28 - protein, 1015 aa, Phytophthora sojae
MTGKCVGIVREVYNKWERRAPLTPAHVRELVQRGIQVLVQPSTARVFSDEQYVRAGATLSEDLAPANVIVGVKQVPEPALLADKTYLFFSHTIKAQPENMALLDAVLQRRITLVDYECITEESGKRLIAFGGNAGRAGMIAGFRGLGERLINMGVSSPFVNVASAYMYSDLEHAKDAVEAAGKRIRSDGLPSELVPMTFAFTGNGNVSKGAQEIFKLMPHEMVHPSELPKLPKNNRILYGTVIDNPDYFVKPQPGFSGTPSRAHYYQNPHQYEPAFHEKVLPYTSMLVNCMYWDDRFPRLVTREQIRELRGSGNHKLLGIADISCDIGGSVEFLERATEIERPFALYDVAEDKMREDGDSRGLEGDDGIMMMGVDILPSELARESSQQFGDRLVGYVTALSSVTSSNVPLHEQKELPAELRGACIASKGVLAPRYEYIHRMRAERERSKQFKFLDAQQEVAGSTCLLLEGHLFDTGLINQVLNLIEDHDGGFHLVDCEVRPNIAAGDSGFSTVSNAIVQVSMSDRAALDDIIAKIRSLADLTSGAKAIVTELPDLCGTNYSKTRGAVVRKDAAANTAADVSVSSPKKRQIVCFGAGLVASPLVEYLSREQGNEVHVVSGIEGEVKGVMRKISRRNIKPHVVNVAEDGAGVDKLCAEADCVVSLLPATMHTTIAQRCIQHGTPLVTASYVSPEMKELDAKAKKAGIPILCEIGLDPGMDHMSAMKVIDEVKAHSGKILSFSSVCGGLPAPEAADNAIGYKFSWSPRGVLTAALNAAQYRKDGKIVNVAGEDLLNSSEPVNFLPAFNIEQIPNRDSLPYGEIYVIPEAHSLYRGTLRYGGCCRILYQLRKLGLFDMDPSKPIPATWPDLISQLGGHQGLREDANGFLQWLGAFDHSTPVVRAPSILDAFCALLQDKLSYQPGERDMAIMHHEFGIEYEDGKKEKRTSTFVGYGSDKGDTIMAKTVGLSAAIGVQLILQDAVQGRGVLTPTTPDIYGPALARLEVEGVRFIEKTFPQP

SEQ ID NO: 29 - DNA, 1164 bp, Pyrenophora tritici-repentis
  CDS (1)..(1164): Lys1
atggctttccccacactacatgcccgcgcagaggctaaacctcttgaacaccgctcgtgtctcacacccacaaccgcaaagaagctgctcgatgctggctaccctgtcctcgtcgagcgctcgcccaaggaccccaactacgcccgtatcttcaaagacgacgagttcgaggaggtaggcgcgacgctcatcgaggaaggcgcatacaagacggcccccaaggaccgcatcatcatcggcctaaaggagctgccagaggacaagttcccgctcgagcacacattcgtacactttgcacattgctacaagcagcaaggcggatgggaaaacgtattggcgaggttcccccgtggcggtggcacattgtatgacctcgagttcctgcaagatgagtcaggcaggcgcgttgctgcctttggctaccacgccggtttcgtcggcgctgctcttgccatcaagacatgggcttggcaactcacccatcccaacggcgagccccttcctggtctagagaccttcactgagggacgcggctactacaacaacgagagcgagttgattacccaactcaaggaagatgtcgcagccggcgaaaagatcgcaggacacaagcccagcagtctggtgctaggtgctctcggccgctgcggctctggtgccgtcgacctccttgagaagattggctgccctgagatcaagaagtgggatcttgccgagaccaaggagcgcgacggcccataccccgaaattgtcgaatctgacatcttcgtcaactgcatctacctttccaagcccatcccgccttttgtcaacaaggagagtctcaagtcgccaaaccgcagactcagcgtcgtttgcgatgtcagctgtgacactactaaccctcataaccccattcccatctacgatatcaacaccacctttgacaagcccacagttgaggtttctgtcgagggtgacggtcctagactatctgtcatctccattgaccaccttccctctgcgttgcctcgtgagtcttcagaagcctttagcaacgctctgctgcctagcttgatggctctgaaggaccgcgcgacaacgcctgtgtggcagggtgctgagaagctcttccaagagaaggttcagacgctgccgggtggtgtccccgcaaaggaggtatag

SEQ ID NO: 30 - protein, 387 aa, Pyrenophora tritici-repentis
MAFPTLHARAEAKPLEHRSCLTPTTAKKLLDAGYPVLVERSPKDPNYARIFKDDEFEEVGATLIEEGAYKTAPKDRIIIGLKELPEDKFPLEHTFVHFAHCYKQQGGWENVLARFPRGGGTLYDLEFLQDESGRRVAAFGYHAGFVGAALAIKTWAWQLTHPNGEPLPGLETFTEGRGYYNNESELITQLKEDVAAGEKIAGHKPSSLVLGALGRCGSGAVDLLEKIGCPEIKKWDLAETKERDGPYPEIVESDIFVNCIYLSKPIPPFVNKESLKSPNRRLSVVCDVSCDTTNPHNPIPIYDINTTFDKPTVEVSVEGDGPRLSVISIDHLPSALPRESSEAFSNALLPSLMALKDRATTPVWQGAEKLFQEKVQTLPGGVPAKEV

SEQ ID NO: 31 - DNA, 1119 bp, Sclerotinia sclerotiorum
  CDS (1)..(1119): Lys1
atgtctggaactactcttcacctacgttctgagttgggaaaagctcttgagcatagatcagctcttacccccactacagccaaagctctcatcgatgctggatacacaattaatgtcgagcgcagcccagaacgtatattcgacgatgaggagttcgagaaggttggagctactctcgtgccagaagatacatggagacaagcaccaaaggatcatattattattggtttgaaggaactacctgtcgaagactttcccctcgagcatgtccacgtacaattcgcacactgttacaaacaacaaggtggctgggacaccgttctatcacgatttcctcgcggcggcggaactctcttagatctcgaatttttgacagacgataaaggcagaagagttgcagctttcggataccacgctggatttgctggtgcagcattggcactcgaaacttgggcatggcaactcactcattcagcatcagagccattcccaagcgtatcaagctatcctaacgaagatgagttgattgccgacgtaaagaaagcaatcgcagctggacaagaaaagacaggcaaggcaccacgagttttggttatcggagcattgggcagatgtggaagtggagcagttgatctctgcttgagagccggtgtgccaaccgagaatgtcttgaagtgggatatggcagagactgccaagggaggaccatttccagagattgtcgagagtgacatcttcattaactgcatatacctcatgtccaagattccgaactttgtcgacatgcaaagcctcgataccccaaaccgcaaattgtcagtcgtctgcgacgtcagtgctgataccacaaatcccaacaatccaattccaatctataccgttgcgaccacattttcagagccaactgtcccagtcgaggtcaagggagaccccagactaagcgtcatcagcattgatcacttaccaagtctattgccaagagaagcaagcgaggcattcagcaaggacttgctaccaagtttgttgacattaaaggactggagaagtactccagtctgggcgaaggccgagaagttgtttcaggagaaggttgccacattgcctaagaaggaggcatga

SEQ ID NO: 32 - protein, 372 aa, Sclerotinia sclerotiorum
MSGTTLHLRSELGKALEHRSALTPTTAKALIDAGYTINVERSPERIFDDEEFEKVGATLVPEDTWRQAPKDHIIIGLKELPVEDFPLEHVHVQFAHCYKQQGGWDTVLSRFPRGGGTLLDLEFLTDDKGRRVAAFGYHAGFAGAALALETWAWQLTHSASEPFPSVSSYPNEDELIADVKKAIAAGQEKTGKAPRVLVIGALGRCGSGAVDLCLRAGVPTENVLKWDMAETAKGGPFPEIVESDIFINCIYLMSKIPNFVDMQSLDTPNRKLSVVCDVSADTTNPNNPIPIYTVATTFSEPTVPVEVKGDPRLSVISIDHLPSLLPREASEAFSKDLLPSLLTLKDWRSTPVWAKAEKLFQEKVATLPKKEA

SEQ ID NO: 33 - DNA, 1170 bp, Trichoderma reesei
  CDS (1)..(1170): Lys1
gttcaactattttccccctcattatcataccacaaaaaacaacaacaatcaattcgtgccaaaatgcctaccgtcatccatctgagagccgataccaagccgtttgagcgtcgctcgccgctgtcccccccaaccgccaaggccctcctcgacgctggctacgtcgtccgcgttgagcgatcttcagagcgcatctacaaggacgaagagttcgaggccgttggcgccgaattggtccctgccggatcatggatcaaggctcccaaggaggacatcatcctgggcctcaaggagctgcccgacgacgacattgacctgcctcacacatacatccacttccagcacatcttcaagaagcagctcggctgggcgccctccctgaagcgctttgctcgcgccggcggcacgctctacgacctggaattcctgactgaggagaacggccgcagaatcgcggcctttggctactttgctggatatgccggcgccgccattgccttcatctcgtgggctcaccagatcctcaaccccggcgtcccccagcccccggtgcctctgttcgacagcgcgcctgctctggtcgcgcatgtcaaggctgcgctcgagcccgcgattcgtgccaacaacggccaacttccccgcgtcattgtgattggcgctctgggacgatgcggcaagggagccgtggacttttgccgcgaggttggtcttcctgaggactccatcctcaagtgggatatgcaggagactgccaagggagggcctttcgaggaaatcaccacttccgacatcttcatcaactgcgtctaccttggccctactcccactcctccctttgtcacattcgaatcgcttgcgaccccagagagacgactccgcgtcattgccgatatctcgtgtgatcccaacagcgagaacaacccgatccccttgtactccacctggtccagctttgacaagcccacgatcccgacatccaagcccgttgacggccctgagctgagaatcattgccatcgaccacctccccaccttgattgcccgcgagtccagcgatgagtactctggcctgctgctgccggccctgctcactctggacaagcgggatactgagggtgtctggacgagagcggagaagacatacaaggacagagtggctgagctgccttag

SEQ ID NO: 34 - protein, 389 aa, Trichoderma reesei
VQLFSPSLSYHKKQQQSIRAKMPTVIHLRADTKPFERRSPLSPPTAKALLDAGYVVRVERSSERIYKDEEFEAVGAELVPAGSWIKAPKEDIILGLKELPDDDIDLPHTYIHFQHIFKKQLGWAPSLKRFARAGGTLYDLEFLTEENGRRIAAFGYFAGYAGAAIAFISWAHQILNPGVPQPPVPLFDSAPALVAHVKAALEPAIRANNGQLPRVIVIGALGRCGKGAVDFCREVGLPEDSILKWDMQETAKGGPFEEITTSDIFINCVYLGPTPTPPFVTFESLATPERRLRVIADISCDPNSENNPIPLYSTWSSFDKPTIPTSKPVDGPELRIIAIDHLPTLIARESSDEYSGLLLPALLTLDKRDTEGVWTRAEKTYKDRVAELP

SEQ ID NO: 35 - DNA, 1173 bp, Ustilago maydis
  CDS (1)..(1173): Lys1
atgtctacaagccgccaacccctctacctccgatgcgagatgaagccggccgagcaccgtgccgcgctcaccccaaccaccgccaaggcactcattgacgctgggttcgacatcacggtcgagtccgaccctcaacgtatctttgacgacaaagagtacaccgaggtagggtgcaagcttgcccctcacaacacgtttcactccctgcccgccgacatccccatcatcggtctcaaggagcttgaggagcctggtccggatctgcctcacacccacattcagtttgctcactgctacaagaagcaggccggttgggcggatgttctgggtcgattcaagcgcggaggcggaaagctctacgatttggagtttttggaggacaagaacggccgtcgtgtagccgcgtttggttggcacgccggctttgcaggcgctgcacttggtctgttggctttggctgaacaggtgcaaggtgaagaccaaaggctgggtgctcaaaaggcttaccccaacgagcaggcgctcatcgcacacgccaagcagcagattgagttcatcaagaaatcgcgctcggacggcaaagtgaaggcgctcgtcgtcggagctttgggacgatgtggtcgtggagccattgactttttcgaaaaggccggcgtcgcttctgaggacattgtccgctgggacatccaggagacctcagccaagcacggtccctaccaagagctgctcgacgtagacatctttgtcaactgcatctacctcacctctaaaatcccgcctttcctcgaccaacctacgatccaagccgctggtccctcgcgtcgtctcggcgtcgtcgtcgacgtatcgtgcgacactaccaaccccaacaaccctctccccatctacgacatcaacaccacctttgacaaacctaccgtcgacgtcaacacgggcaaaggtaacccgagcttgaccgtcatctcgatcgaccatctgcccactctgctgcctagggagagctccgagggtttcagcaacgacctgttgcccagtctgttgcagttgccctacgtgctcggtaaggacaccaccaagctcgacacgttggacgaaggaaagggagccgtctggcagagagccgaaaagctcttccataagcacttggcagaggctgagcagcacacagcttga

SEQ ID NO: 36 - protein, 390 aa, Ustilago maydis
MSTSRQPLYLRCEMKPAEHRAALTPTTAKALIDAGFDITVESDPQRIFDDKEYTEVGCKLAPHNTFHSLPADIPIIGLKELEEPGPDLPHTHIQFAHCYKKQAGWADVLGRFKRGGGKLYDLEFLEDKNGRRVAAFGWHAGFAGAALGLLALAEQVQGEDQRLGAQKAYPNEQALIAHAKQQIEFIKKSRSDGKVKALVVGALGRCGRGAIDFFEKAGVASEDIVRWDIQETSAKHGPYQELLDVDIFVNCIYLTSKIPPFLDQPTIQAAGPSRRLGVVVDVSCDTTNPNNPLPIYDINTTFDKPTVDVNTGKGNPSLTVISIDHLPTLLPRESSEGFSNDLLPSLLQLPYVLGKDTTKLDTLDEGKGAVWQRAEKLFHKHLAEAEQHTA

SEQ ID NO: 37 - DNA, 1137 bp, Verticillium albo-atrum
  CDS (1)..(1137): Lys1
atgtctggcatcaccctacacctccggtccgagaccaagccgctggagcaccgcagtgctctgacccccttcacggcatccgagctgatcaaggctggctacaccctcaatgtcgagcgcagccctgttcgcatcttcgacgatgccgagttcgagaagattggcgccaccctcgtccccgagggtagctggaccgaagccccccaagaccacatcattgttggcctcaaggagcttcttgaggaggatttccctctgaagcacgttcacgtccagtttgcgcactgctacaagcagcagggcgggtgggagaacgtcctggcccgcttccctcgtggtggcggcaccctctacgacctcgaattcctcgagaaggaggtcgcccccggccgctttgcccgagtcgccgcctttggctggagcgccggtttctctggtgctgctctggctctccagaactgggcctggcagctcaacaaccccggcaagcccctgccttcggtcgagagctatcccaacgaggacgagctcatcactgctgtcaagaagagcattgccgagggcaaggagaaggccggcaagctaccccaggtcctcgtgatcggcgccctcggccgctgcggcagcggtgccgtcgagctgtgccgccgcgtcggcctgcccgaggagcagatcctcaagtgggatctcgaggagacgaagaagggcggccccttcaccgagattgttgagagcgacatctttgtcaactgcatctacctcaacagcaagattcccaactttgtcgactttgagtctctcaagtcgcccaagaggcagctgtccgtcgtctgcgacgtctctgctgacaccaccaaccccaacaaccctgtccccatctacaccgtcgccacaaccttcgacaagcccacagtgcccgtcgagggactcgagaacccgcccctgagcgtcatcagcattgatcaccttcccagcctcctcccccgtgaggcctccgagacgtacagcaaggacctgctgccgtacctgctcacgctgaaggaccgcaagacggaccccgtctggacgagggctgagaagctcttcaacgacaaggtcgctacgctgccggccgacctgcagaagcctgctcagtga

SEQ ID NO: 38 - protein, 378 aa, Verticillium albo-atrum
MSGITLHLRSETKPLEHRSALTPFTASELIKAGYTLNVERSPVRIFDDAEFEKIGATLVPEGSWTEAPQDHIIVGLKELLEEDFPLKHVHVQFAHCYKQQGGWENVLARFPRGGGTLYDLEFLEKEVAPGRFARVAAFGWSAGFSGAALALQNWAWQLNNPGKPLPSVESYPNEDELITAVKKSIAEGKEKAGKLPQVLVIGALGRCGSGAVELCRRVGLPEEQILKWDLEETKKGGPFTEIVESDIFVNCIYLNSKIPNFVDFESLKSPKRQLSVVCDVSADTTNPNNPVPIYTVATTFDKPTVPVEGLENPPLSVISIDHLPSLLPREASETYSKDLLPYLLTLKDRKTDPVWTRAEKLFNDKVATLPADLQKPAQ

SEQ ID NO: 39 - DNA, 1125 bp, Mycosphaerella graminicola
  CDS (1)..(1125): Lys1
atgtcttcttcaccactaactttgcacatccgcgcggaaaccaagcccctcgaacatcgcaccgctgttccgcccaaggttgcgaggaaacttgtcgaggcgggttacgtggtcaacgtcgagcggagtccattgagcatcttcccggacaacgagtacgaaggaacaggagctacgcttgtgccgactggttcttggacggaagcacccaaggaccatatcgttgtgggattgaaggagttgccggaggaggatttcgcgttggtgcatacgcatgttcagttcgcacattgctataagaatcagggcggatgggagaaagtcttgagtcgattcccgagaggtggagggacgctgcttgacttggagttcctcgaagatgagcaaggacgacgagttgcggctttcggctaccacgccggattcgcaggagcagctctctccctcatcacatgggcctggcaactcgagcacggcacctccaagcccgtgcctggagtcaccgcctacgagaacgaaacactcctcgtcaacgacgtgaagaaagccgtcgagaagggcaagtcgatcgccggccaccttccgcgagttctggtcatcggcgctctcggacgttgtggccgtggagccgtcgacctctgcgtcaaagctggtctgcaagacatcctcaaatgggacctgcaagagaccaaagccaaaccaggcccctaccaagaaatcatcgagtccgacgtctttgtaaactgcatctacctctccgccaaaatccctccattcatcgacgcaccttccctcgcctccccgacccgcaaactcagcgtcgtctgtgacgtctcctgcgacaccacgaatccacacaatccgattccgatttactccatcaatacgacgtttgacaagcccaccgtgcctgtggaactgtcctcagaggcgaacgatgtgccgttgagtgtgattagcatcgaccacttgccgagtttgttgccgagagaggcgagcgaggcattcagcgaggcattgttgccgagtttgttggagttgaaggagaggaagacggcgcgggtgtggaggcaggcggagaagttgttcgaggacaaggtggctagtctgccgaagggttcgtac

SEQ ID NO: 40 - protein, 375 aa, Mycosphaerella graminicola
MSSSPLTLHIRAETKPLEHRTAVPPKVARKLVEAGYVVNVERSPLSIFPDNEYEGTGATLVPTGSWTEAPKDHIVVGLKELPEEDFALVHTHVQFAHCYKNQGGWEKVLSRFPRGGGTLLDLEFLEDEQGRRVAAFGYHAGFAGAALSLITWAWQLEHGTSKPVPGVTAYENETLLVNDVKKAVEKGKSIAGHLPRVLVIGALGRCGRGAVDLCVKAGLQDILKWDLQETKAKPGPYQEIIESDVFVNCIYLSAKIPPFIDAPSLASPTRKLSVVCDVSCDTTNPHNPIPIYSINTTFDKPTVPVELSSEANDVPLSVISIDHLPSLLPREASEAFSEALLPSLLELKERKTARVWRQAEKLFEDKVASLPKGSY

SEQ ID NO: 41 - DNA, 1170 bp, Fusarium moniliforme
  CDS (1)..(1170): Lys1
atgtctgactatccccacattctccttcgcgctgaggagaagcctctcgagcaccgatctttctcccccgcaattatcaagacactcgttgatgctggataccccatttccgtcgagcgatcgtctaccgaccccaagttcaagcgtatctttgaggactcagaatatgaggctgctggtgctcgtcttgtcgatacgggtgtctggcccaacgctgagcctggaacaatcattctcggcctaaaagagctaccttctgaggacttcccgctcaagaatgaccacattacatttgcacattgttacaagaaccaaggcgggtgggagcaggtcctcggtcgctgggcacgcggtggcagccggctttacgacctcgagtttcttgtggacgaacaaggtcggcgtgtttctgcatttgggtaccacgcgggcttcgccggtgctgcgctcggaatcaagacactcgctcaccagctgcagggctcgtcctctaaacttccttctgtcgagacattcactgatggccgcggatattacttgaacgaagatgagctcgtcaaccagattcgtgaggatctcgctaaggctgaaaaggctctcggacgtaagcccactgctctcgtccttggtgctcttggacgatgtggtaagggtgccgtggaccttttcctgaaggccggcatgcctgatgacaacattacccgctgggacttgaacgagactaaggaccgtgatggcccttacgaggagatcgccaaggctgatgtcttcctcaacgccatctacctctccaagcccattccccctttcatcaaccaagaactccttgccaagcaaggtcgcaacctcgctgttgtcatcgacgtttcttgtgacaccacaaaccctcacaaccctatccccatctactccatcaacaccacctttgaggacccaaccgtccccgttgagatcaaggacgatcagaacaacctccccctatccgtcatcagcattgatcacctcccttccatgcttccccgcgaggctagtgaggcctttagtgagggtctcaaggagtctctgctcacactcaaggatcgcgagacttcgcgggtgtggactgatgctgagaagctcttccatgagaaggttgccctgttgcccgaggagttgagaaccaagagtgtt

SEQ ID NO: 42 - protein, 390 aa, Fusarium moniliforme
MSDYPHILLRAEEKPLEHRSFSPAIIKTLVDAGYPISVERSSTDPKFKRIFEDSEYEAAGARLVDTGVWPNAEPGTIILGLKELPSEDFPLKNDHITFAHCYKNQGGWEQVLGRWARGGSRLYDLEFLVDEQGRRVSAFGYHAGFAGAALGIKTLAHQLQGSSSKLPSVETFTDGRGYYLNEDELVNQIREDLAKAEKALGRKPTALVLGALGRCGKGAVDLFLKAGMPDDNITRWDLNETKDRDGPYEEIAKADVFLNAIYLSKPIPPFINQELLAKQGRNLAVVIDVSCDTTNPHNPIPIYSINTTFEDPTVPVEIKDDQNNLPLSVISIDHLPSMLPREASEAFSEGLKESLLTLKDRETSRVWTDAEKLFHEKVALLPEELRTKSV

SEQ ID NO: 43 - DNA, 856 bp, Claviceps purpurea
  CDS (3)..(854): Lys1
ctggctacaaagtttccgtggagcgctcaccatcggaccccgactttgtccgcatcttccaagactctgaatatgaggcggctggagcttctctggttcccactggcgtgtggcccaaggctgcgccaaacactctgatcgttggtctcaaggagatccccgaagagggcttttctctcaccaatgaccacctgacctttgcccattgctacaagaagcaagctggctggacacaggttctcggacgctttgtcgcaggcaaatcgaccctctacgacctggagtttctggtcgatagcactggaagacgcatctctgctttcggattccacgctggcttcacgggcgctgctttgggagtgaagaactgggcttggcaactggccaatccagccaagaagcttcccgccgtgtccacattcactgacggcaagggatactatgtgaacgaaaccgagttggtggagcaaatccgcaaggatctcgaggcaggagagaagattcttggccgtaagccaacagcctttgtcctgggagctttgggtcgctgcggcaggggtgcttgcgatttgttcctcaaggcaggcttgcccgaagagaacatcacccgatgggatttggccgaaacccgcgaccgccaaggcccgtacgaggagattgctcagcacgatatcttcctgaacgctatctatctctccgagcccatcccaccttttgtcaacaacgagcttctttctaagcctggccgcaaattgagtgttgtcatagacgtctcctgcgacaccaccaacccccacaaccccatccccatctacagcatcaacaccaccttcgacagccccaccgttgccgtcaa

SEQ ID NO: 44 - protein, 284 aa, Claviceps purpurea
GYKVSVERSPSDPDFVRIFQDSEYEAAGASLVPTGVWPKAAPNTLIVGLKEIPEEGFSLTNDHLTFAHCYKKQAGWTQVLGRFVAGKSTLYDLEFLVDSTGRRISAFGFHAGFTGAALGVKNWAWQLANPAKKLPAVSTFTDGKGYYVNETELVEQIRKDLEAGEKILGRKPTAFVLGALGRCGRGACDLFLKAGLPEENITRWDLAETRDRQGPYEEIAQHDIFLNAIYLSEPIPPFVNNELLSKPGRKLSVVIDVSCDTTNPHNPIPIYSINTTFDSPTVAV

SEQ ID NO: 45 - DNA, 41 bp, Artificial Sequence
  primer
  primer_bind (1)..(41)
taatacgactcactatagggttgcaggagagcgcagaaagc

SEQ ID NO: 46 - DNA, 42 bp, Artificial Sequence
  primer
  primer_bind (1)..(42)
taatacgactcactatagggtcagttggagtccgcgtggtgt

SEQ ID NO: 47 - DNA, 20 bp, Artificial Sequence
  primer
  primer_bind (1)..(20)
cgactctggtgacggtgtgt

SEQ ID NO: 48 - DNA, 20 bp, Artificial Sequence
  primer
  primer_bind (1)..(20)
gcgtgaggaagagcgtaacc

SEQ ID NO: 49 - DNA, 18 bp, Artificial Sequence
  primer
  primer_bind (1)..(18)
ccgcccagacaatttcgt

SEQ ID NO: 50 - DNA, 20 bp, Artificial Sequence
  primer
  primer_bind (1)..(20)
ccttggcccagttgttacca

SEQ ID NO: 51 - DNA, 21 bp, Artificial Sequence
  primer
  primer_bind (1)..(21)
tgggtggtttccaaggtcttc

SEQ ID NO: 52 - DNA, 20 bp, Artificial Sequence
  primer
  primer_bind (1)..(20)
aaaggcaccaagccactgaa

SEQ ID NO: 53 - DNA, 48 bp, Artificial Sequence
  primer
  primer_bind (1)..(48)
tcgacaggcctggatccttaattaaactagtctcgaggagctcggtac

SEQ ID NO: 54 - DNA, 40 bp, Artificial Sequence
  primer
  primer_bind (1)..(40)
cgagctcctcgagactagtttaattaaggatccaggcctg

SEQ ID NO: 55 - DNA, 31 bp, Artificial Sequence
  primer
  primer_bind (1)..(31)
tatctcgagtctagacaacgccattggttac

SEQ ID NO: 56 - DNA, 25 bp, Artificial Sequence
  primer
  primer_bind (1)..(25)
agaggtaccaagcttgcgtagctgg

SEQ ID NO: 57 - DNA, 22 bp, Artificial Sequence
  primer
  primer_bind (1)..(22)
tcaatagaagcgaacgcgtaaa

SEQ ID NO: 58 - DNA, 20 bp, Artificial Sequence
  primer
  primer_bind (1)..(20)
gttcgggatctgctcgatgt

(19) United States
(12) Patent Application Publication
(10) Pub. No.: US 2019/0382770 A1
(43) Pub. Date: Dec. 19, 2019

(54) RNAi FOR THE CONTROL OF FUNGI AND OOMYCETES BY INHIBITING SACCHAROPINE DEHYDROGENASE GENE

(71) Applicant: BASF AGRICULTURAL SOLUTIONS SEED, US LLC, Research Triangle Park, NC (US)

(72) Inventors: Thomas Delebarre, Caluire-et-Cuire (FR); Cécile Dorme, Lyon (FR); Bernd Essigmann, Saint-Didier-au-Mont-d'Or (FR); Frédéric Schmitt, Saint-Didier-de-Formans (FR); François Villalba, Albigny-sur-Saone (FR); Eric Paget, Caluire-et-Cuire (FR)

(21) Appl. No.: 16/422,585

(22) Filed: May 24, 2019

Related U.S. Application Data
(62) Division of application No. 14/349,462, filed on Apr. 3, 2014, now abandoned.
(60) Provisional application No. 61/661,062, filed on Jun. 18, 2012.

(30) Foreign Application Priority Data
Oct. 4, 2011 (EP) 11356013.0

(51) Int. Cl. C12N 15/113; A01N 43/16; A61K 31/713

(52) U.S. Cl. CPC C12N 15/1137; A61K 31/713; A01N 43/16

(57) ABSTRACT

The present invention relates to control of plant pathogens, particularly fungi or oomycetes, by inhibiting one or more biological functions, particularly by inhibiting saccharopine dehydrogenase gene(s) using RNA interference. The invention provides methods and compositions using RNA interference of plant pathogens target genes for such control. The invention is also directed to methods for making transgenic plants tolerant to said plant pathogens, and to transgenic plants and seeds generated thereof.

### CROSS-REFERENCE TO RELATED APPLICATIONS

This application is a divisional of U.S. patent application Ser. No. 14/349,462, filed Apr. 3, 2014, which is a National Stage Entry of PCT/EP2012/069521, filed Oct. 3, 2012, which claims priority to European Patent Application No. 11356013.0 filed on Oct. 4, 2011, and U.S. Provisional Application No. 61/661,062, filed Jun. 18, 2012, the disclosures of each of which are hereby incorporated by reference in their entireties.

### REFERENCE TO SEQUENCE LISTING SUBMITTED ELECTRONICALLY

The Sequence Listing associated with this application is filed in electronic format via EFS-Web and hereby incorporated by reference into the specification in its entirety. The name of the text file containing the Sequence Listing is 5500 185_US_Sequence_Listing. The size of the text file is 229 KB, and the text file was created on Mar. 30, 2014.

The present invention relates to control of plant pathogens and pests, particularly fungi or oomycetes, by inhibiting one or more biological functions, particularly by inhibiting fungi saccharopine dehydrogenase gene involved in the α-aminoadipate pathway for lysine biosynthesis and their oomycetes homologs using RNA interference. The invention provides methods and compositions using RNA interference of fungi or oomycetes target genes for such control. The invention is also directed to methods for making transgenic plants tolerant to said fungi or oomycetes, and to transgenic plants and seeds generated thereof.

The technology used in the context of the present invention is RNA interference or RNAi.

The expression in an organism of a sequence homologous to a target-gene capable of inducing the formation of small double-stranded RNA (dsRNA) makes it possible, very specifically, to extinguish this gene and to observe the phenotype that results therefrom (Xiao et al, 2003).

The dsRNA triggers the specific degradation of a homologous RNA only in the region of identity with the dsRNA (Zamore et al., 2000; Tang et al., 2003). The dsRNA is an RNA molecule which contains a double-stranded sequence, generally of at least 19 base pairs (bp) including a sense strand and an antisense strand. The dsRNA molecules are also characterized by the very large degree of complementarity between the two complementary RNA strands. The dsRNA is degraded into RNA fragments of generally 18 to 25 nucleotides (siRNA) and the cleavage sites on the target RNA are evenly spaced apart by 18 to 25 nucleotides. The small siRNAs resulting therefrom exhibit a very high degree of identity with respect to the target RNA; however, mismatches of 3 to 4 nucleotides between the siRNA and the corresponding portion of the target RNA nevertheless make it possible for the system to operate (Tang et al., 2003). It has thus been suggested that these fragments of 18 to 25 nucleotides constitute RNA guides for recognition of the target (Zamore et al., 2000). These small RNAs have also been detected in extracts prepared from Schneider 2 cells of Drosophila melanogaster which had been transfected with dsRNAs before cell lysis (Hammond et al., 2000). The guiding role of the fragments of 18 to 25 nucleotides in the cleavage of the mRNAs is supported by the observation that these fragments of 19 to 25 nucleotides isolated from dsRNA are capable of being involved in the degradation of mRNA (Zamore et al., 2000). Sizable homologous RNA molecules also accumulate in plant tissues which undergo the PTGS phenomenon (Post Transcriptional Gene Silencing, Hamilton and Baulcome, 1999). These small RNAs can regulate gene expression at three different levels:
transcription (TGS for Transcriptional Gene Silencing),
messenger RNA degradation (PTGS for Post Transcriptional Gene Silencing).
miRNA pathway
translation.

Regulation involving messenger RNA degradation appears to exist in all eukaryotes, whereas regulation at the transcriptional level has only been described in mammalians, plants, drosophila and C. elegans. As regards the regulation of translation, it has been characterized in C. elegans and drosophila and appears also to exist in mammals (Hannon, 2002) and plants (Ruiz Ferrer and Voinnet, 2009). In the literature, reference is made to RNAi, to PTGS, to cosuppression or to quelling (reserved for fungi) when referring to this phenomenon, depending on the organisms in which it is studied. RNAi has in particular proved that it is effective when double-stranded RNA (dsRNA) is injected into the nematode Caenorhabditis elegans (Fire et al. 1998; Montgomery et al., 1998; WO99/32619). Inhibition of the expression of an insect target gene was also observed when this insect is fed with bacteria expressing small double-stranded RNAs corresponding to said insect target gene (WO 01/37654).

More recently, pharmaceutical compositions comprising dsRNA substantially complementary to at least part of a gene suspected to be involved in the human papilloma virus (HPV) infection together with a pharmaceutically acceptable carrier have been disclosed to treating said HPV infection (W02009/0247607).

The introduction of dsRNA was carried out in plants in order to induce silencing of an endogenous target gene (Hamilton et al., 1998, WO99/15682), to induce resistance to RNA viruses by means of the use of a transgene expressing a dsRNA having substantial identity with respect to the viral genes (Waterhouse et al., 1998; Pandolfini et al., 2003, WO98/36083, WO99/15682, U.S. Pat. No. 5,175,102), but also to induce resistance to nematodes (Chuang and Meyerowitz, 2000, WO01/96584) or alternatively to the bacterium Agrobacterium (WO00/26346, Escobar et al., 2001). More recently, it has been shown that plants expressing dsRNA having substantial identity against a fungal gene essential to the growth of the fungus or to its pathogenicity may also induced resistance to this fungus (WO05/071091).

Nevertheless, since that time, only few preliminary results and no commercial examples exist on RNAi-mediated resistance or tolerance to phytopathogenic fungi where the double-stranded (dsRNA) or small interfering (siRNA) molecules are expressed in the plant, or applied as part of an external composition to the seed, the plant or to the fruit of the plant or to soil or to inert substrate wherein the plant is growing or wherein it is desired to grow.

Among others, one difficulty is to find an appropriate target gene, whose inhibition by dsRNA or siRNA induces a good level of fungi tolerance, up to a level suitable for a commercial use, without deleterious effect on the plant expressing said dsRNA or siRNA or on which a composition comprising said dsRNA or siRNA is applied.

Stärkel C. attempted in her Ph. D. thesis [“Host induced gene silencing—strategies for the improvement of resistance against Cercospora beticola in sugar beet (B. vulgaris L.) and against Fusarium graminearum in wheat (T. aestivum L.) and maize (Z. mays L.)”, defended in June 2011] to inhibit the growth of Fusarium graminearum by transforming wheat with silencing constructs targeting the homoaconitase gene, an essential gene in the lysine biosynthesis pathway. Nevertheless, no transgenic wheat plants could be generated. Moreover, the attempt to delete or silence the homoaconitase gene in Fusarium graminearum by transformation of the fungus with a construct designed for the inducible silencing of the homoaconitase gene were also unsuccessful.

The present inventors have surprisingly demonstrated that inhibition of fungus or oomycetes saccharopine dehydrogenase gene, which is involved in the α-aminoadipate (AAA) pathway, via RNAi methodology causes cessation of infection, growth, development, reproduction and/or pathogenicity, and eventually results in the death of the organism.

This new target for the RNAi technology is particularly suitable, considering that AAA pathway is specifically found in some plant pathogens, including higher fungi, and not in plants, humans and animals.

Among the 20 common proteinogenic amino acids, L-lysine is the only one known to have a biosynthetic pathway which differs in plants and in higher fungi. In plants and bacteria, L-lysine is obtained through the diaminopimelate (DAP) pathway. In higher fungi and euglenoids, L-lysine is obtained through the α-aminoadipate (AAA) pathway. Saccharopine dehydrogenase, homocitrate synthase, homoaconitase, homoisocitrate dehydrogenase, α-Aminoadipate aminotransferase, α-Aminoadipate reductase and saccharopine reductase are enzymes involved in the α-aminoadipate pathway for the biosynthesis of L-lysine.

None of the enzymes involved in these two distinct pathways (DAP and AAA pathways) are common (Xu et al., 2006, Bhattacharjee, J. K., 1985; Bhattacharjee, J. K., 1992; Vogel, H. J., 1965). As an example, fungi saccharopine dehydrogenase is involved in the Lysine biosynthesis, when the lysine-ketoglutarate from plant, sometimes also called saccharopine dehydrogenase, is responsible for lysine catabolism (Houmard et al., 2007). For humans and animals, L-lysine is an essential amino acid which can only be obtained from protein in the diet. Additionally, enzymes involved in the fungal AAA pathway are unique to lysine synthesis (Umbargar, H. E., 1978; Bhattacharjee, J. K., 1992).

The presence of a specific α-aminoadipate (AAA) pathway in higher fungi, which is not present nor in plant nor in humans or animals, leads to consider the enzymes involved in said AAA pathway as particularly attractive targets for the control of plant pathogens, particularly for fungal pathogens.

Interestingly, although lysine has been reported to be synthesized in oomycetes via the diaminopimelic pathway (Born and Blanchard, 1999), genes homologous to fungus genes of AAA pathway have been found in oomycetes, which may indicate that both pathways might be present in oomycetes. The inventors have shown that dsRNA against oomycete saccharopine dehydrogenase gene substantially reduced the growth of said oomycete, and that plants expressing dsRNA against oomycete saccharopine dehydrogenase genes could be less susceptible to said oomycete infection.

The presence of the AAA pathway for lysine biosynthesis has been demonstrated and studied for example in Saccharomyces cerevisiae (Broquist, H. P., 1971, Bhattacharjee, J. K., 1992), the human pathogenic fungi Candida albicans (Garrad, R. C. and Bhattacharjee, J. K., 1992), and the plant pathogen Magnaporthe grisea (Umbargar, H. E., 1978). The saccharopine dehydrogenase enzymes involved in the AAA pathway have been intensively studied and compared in different organisms, and their technical features, including their nucleotides and aminoacids sequences, kinetics, substrate specificity, function, 3D-structure, as well as the way to purify and characterize them, are well known from the skilled man (see Xu et al., 2001, the content of which is incorporated herein by way of reference). Numerous genes from different fungi or oomycetes and their sequence data are disclosed and available in searchable public database, such as genBank.

Randall, T. A. et al. (2005) reports a list of genes of AAA pathway from the oomycete Phytophthora infestans, identified by their accession numbers and the sequence data thereof, incorporated herein by reference, are available in searchable public database (s) (Randall, T. A., 2005, MPMI, 18, 229-243; see in particular table 8 p 239).

In one embodiment, the present invention provides a dsRNA molecule comprising 1) a first strand comprising a sequence substantially identical to at least 18 contiguous nucleotides of a fungus or oomycete gene and ii) a second strand comprising a sequence substantially complementary to the first strand, wherein said fungus or oomycete gene is a saccharopine dehydrogenase gene.

As used herein, “RNAi” or “RNA interference” refers to the process of sequence-specific gene silencing, mediated by double-stranded RNA (dsRNA). As used herein, “dsRNA” or “dsRNA molecule” refers to RNA that is partially or completely double stranded. Double stranded RNA is also referred to small or short interfering RNA (siRNA), short interfering nucleic acid (siNA), short interfering RNA, micro-RNA (miRNA), circular interfering RNA (ciRNA), short hairpin RNA (shRNA) and the like.

As used herein, taking into consideration the substitution of uracil for thymine when comparing RNA and DNA sequences, the term “substantially identical” or “essentially homologous” as applied to dsRNA means that the nucleotide sequence of one strand of the dsRNA is at least about 80%, at least 85% identical to 18 or more contiguous nucleotides of the target gene, more preferably at least about 90% identical to 18 or more contiguous nucleotides of the target gene, and most preferably at least about 95%, 96%, 97%, 98% or 99% identical or absolutely identical to 18 or more contiguous nucleotides of the target gene. 18 or more nucleotides means a portion, being at least about 18, 20, 21, 22, 23, 24, 25, 50, 100, 200, 300, 400, 500, 1000, 1500, or 2000 consecutive bases or up to the full length of the target gene.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. As used herein, the term “substantially complementary” means that two nucleic acid sequences are complementary over at least 80% of their nucleotides. Preferably, the two nucleic acid sequences are complementary over at least 85%, 90%, 95%, 96%, 97%, 98%, 99% or more or all of their nucleotides. Alternatively, “substantially complementary” means that two nucleic acid sequences can hybridize under high stringency conditions. As used herein, the term “substantially identical” or “corresponding to” means that two nucleic acid sequences have at least 80% sequence identity. Preferably, the two nucleic acid sequences have at least 85%, 90%, 95%, 96%, 97%, 98%, 99% or 100% of sequence identity.

Also as used herein, the terms “nucleic acid” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, locked nucleic acid or the 2′-hydroxy in the ribose sugar group of the RNA can also be made.

Accordingly to the invention, the first strand and second strand may have identical sizes. Alternatively, the size of the first strand may be greater than that of the second strand. By way of example, the size of the first strand can be about 200 nucleotides greater than the size of the second strand. In another aspect of the invention, the size of second strand is greater than that of the first strand.

Accordingly to the invention, the dsRNA molecule comprises a first strand comprising a sequence substantially identical to at least 18 contiguous nucleotides of a fungus or oomycete saccharopine dehydrogenase gene.

In a particular embodiment, the invention provides a dsRNA molecule comprising 1) a first strand comprising a sequence substantially identical to at least 18 contiguous nucleotides of a fungus or oomycete gene and ii) a second strand comprising a sequence substantially complementary to the first strand, wherein said fungus or oomycete gene is selected from the list consisting of:

a) a polynucleotide comprising a sequence as set forth in SEQ ID NO: 1, 3, 5, 7, 9, 11, 13, 15, 17, 19, 21, 23, 25, 27, 29, 31, 33, 35, 37, 39, 41, 43;
b) a polynucleotide encoding a polypeptide having a sequence as set forth in SEQ ID NO: 2, 4, 6, 8, 10, 12, 14, 16, 18, 20, 22, 24, 26, 28, 30, 32, 34, 36, 38, 40, 42, 44;
c) a polynucleotide having at least 70% sequence identity, preferably at least 80%, more preferably at least 90%, even more preferably at least 95% to a polynucleotide having a sequence as set forth in SEQ ID NO: 1, 3, 5, 7, 9, 11, 13, 15, 17, 19, 21, 23, 25, 27, 29, 31, 33, 35, 37, 39, 41, 43;
d) a polynucleotide encoding a polypeptide having at least 70% sequence identity, preferably at least 80%, more preferably at least 90%, even more preferably at least 95%, to a polypeptide having a sequence as set forth in SEQ ID NO: 2, 4, 6, 8, 10, 12, 14, 16, 18, 20, 22, 24, 26, 28, 30, 32, 34, 36, 38, 40, 42, 44:
e) a polynucleotide hybridizing under stringent conditions to a polynucleotide having a sequence as set forth in SEQ ID NO: 1, 3, 5, 7, 9, 11, 13, 15, 17, 19, 21, 23, 25, 27, 29, 31, 33, 35, 37, 39, 41, 43; and
f) a polynucleotide hybridizing under stringent conditions to a polynucleotide encoding a polypeptide having at least 70% sequence identity to a polypeptide having a sequence as set forth in SEQ ID NO: 2, 4, 6, 8, 10, 12, 14, 16, 18, 20, 22, 24, 26, 28, 30, 32, 34, 36, 38, 40, 42, 44.

In accordance with the present invention, the term “identity” is to be understood to mean the number of amino acids/nucleotides corresponding with the amino acids/nucleotides of other protein/nucleic acid, expressed as a percentage. Identity is preferably determined by comparing the Seq. ID disclosed herein with other protein/nucleic acid with the help of computer programs. If sequences that are compared with one another have different lengths, the identity is to be determined in such a way that the number of amino acids, which have the shorter sequence in common with the longer sequence, determines the percentage quotient of the identity. Preferably, identity is determined by means of the computer program ClustalW, which is well known and available to the public (Thompson et al., 1994). ClustalW is made publicly available on

http://www.ebi.ac.uk/tools/clustalW2/index.html.

Preferably, Version 2.1 of the ClustalW computer program is used to determine the identity between proteins according to the invention and other proteins. In doing so, the following parameters must be set: KTUPLE=1, TOPDIAG=5, WINDOW=5, PAIRGAP=3, GAPOPEN=10, GAPEXTEND=0.05, GAPDIST=8, MAXDIV=40, MATRIX=GONNET, ENDGAPS(OFF), NOPGAP, NOHGAP.

Preferably, Version 2.1 of the ClustalW computer program is used to determine the identity between the nucleotide sequence of the nucleic acid molecules according to the invention, for example, and the nucleotide sequence of other nucleic acid molecules. In doing so, the following parameters must be set:

KTUPLE=2, TOPDIAGS=4, PAIRGAP=5, DNAMATRIX:IUB, GAPOPEN=10, GAPEXT=5, MAXDIV=40, TRANSITIONS: unweighted.

In accordance with the present invention, the term ‘hybridizing under stringent conditions” refers to polynucleotides or nucleic acid sequences which hybridize with a reference nucleic acid sequence at a level significantly greater than the background noise. The background noise may be associated with the hybridization of other DNA sequences present, in particular of other cDNAs present in a cDNA library. The level of the signal generated by the interaction between the sequence capable of selectively hybridizing and the sequences defined by the sequence IDs above according to the invention is generally 10 times, preferably 100 times, greater than that of the interaction of the other DNA sequences generating the background noise. The level of interaction can be measured, for example, by labeling the probe with radioactive elements such as ³²P. The selective hybridization is generally obtained by using very severe conditions for the medium (for example 0.03 M NaCl and 0.03 M sodium citrate at approximately 50° C.-60° C.). The hybridization can of course be carried out according to the usual methods of the state of the art (in particular Sambrook et al., 2001, Molecular Cloning: A Laboratory Manual, third edition).

In a particular embodiment of the invention, the dsRNA molecule is applied on the plant pathogen, particularly fungi or oomycete, and/or on the plant or crop to be protected. The present invention therefore also relates to a composition comprising an effective and non-phytotoxic amount of a dsRNA molecule as defined herein.

dsRNA molecules accordingly to the invention may be made by classical chemical synthesis, by means of in vitro transcription or produced in organisms like animals cells, bacteria, yeasts, or plants by heterologous expression (Aalto A. P. et al, 2007 RNA. 13(3):422-9.)

The present invention therefore relates to a micro-organism producing a dsRNA molecule as herein defined.

The present invention also relates to a genetic construct which comprises at least one DNA sequence as well as heterologous regulatory element(s) in the 5′ and optionally in the 3′ positions, characterized in that the DNA sequence is able to form a dsRNA molecule as herein defined.

The present invention also relates to a cloning and/or expression vector, characterized in that it contains at least one genetic construct as herein defined.

The expression “effective and non-phytotoxic amount” means an amount of composition according to the invention which is sufficient to control or destroy the pathogen present or liable to appear on the crops and which does not entail any appreciable symptom of phytotoxicity for the said crops. Such an amount can vary within a wide range depending on the pathogen to be controlled, the type of crop, the climatic conditions and the compounds included in the composition according to the invention. This amount can be determined by systematic field trials, which are within the capabilities of a person skilled in the art.

Thus, according to the invention, there is provided a composition comprising, as an active ingredient, an effective amount of a dsRNA molecule as herein defined and an agriculturally acceptable support, carrier, filler and/or surfactant.

According to the invention, the term “support” denotes a natural or synthetic organic or inorganic compound with which the active compound of formula (I) is combined or associated to make it easier to apply, notably to the parts of the plant. This support is thus generally inert and should be agriculturally acceptable. The support can be a solid or a liquid. Examples of suitable supports include clays, natural or synthetic silicates, silica, resins, waxes, solid fertilisers, water, alcohols, in particular butanol organic solvents, mineral and plant oils and derivatives thereof. Mixtures of such supports can also be used.

The composition according to the invention can also comprise additional components such as, but not limited to, surfactant, protective colloids, adhesives, thickeners, thixotropic agents, penetration agents, stabilisers, sequestering agents. More generally, the active compounds can be combined with any solid or liquid additive, which complies with the usual formulation techniques.

In general, the composition according to the invention can contain from 0.05 to 99% by weight of active compound, preferably 10 to 70% by weight.

Compositions according to the invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure),gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. These compositions include not only compositions which are ready to be applied to the plant or seed to be treated by means of a suitable device, such as a spraying or dusting device, but also concentrated commercial compositions which must be diluted before application to the crop.

The compounds according to the invention can also be mixed with one or more phytopharmaceutical or plant growth promoting compound, such as a fungicide, herbicide, insecticide, nematicide, acaricide, molluscicide, resistance inducer, safeners, signal compounds, biologicals, pheromone active substance or other compounds with biological activity. The mixtures thus obtained have a broadened spectrum of activity. The mixtures with other fungicide compounds are particularly advantageous.

In a particular embodiment of the invention, the dsRNA is introduced or produced into the plant to be protected. After introduction or production into the plant, the dsRNA may further be processed into relatively small fragments (siRNAs) and can subsequently become distributed throughout the plant. Alternatively, the dsRNA is introduced or produced into the plant using a regulatory element or promoter that results in expression of the dsRNA in a tissue, temporal, spatial or inducible manner and may further be processed into relatively small fragments by a plant cell containing the RNAi processing machinery.

The invention therefore relates to a genetic construct or chimeric gene which is able to produce the dsRNA of the invention inside a plant cell. Said genetic construct or chimeric gene comprises at least one DNA sequence as well as well as heterologous regulatory element(s) in the 5′ and optionally in the 3′ positions which are able to function in a plant, characterized in that the DNA sequence(s) is (are) able to form a dsRNA molecule as herein defined once expressed in the plant.

In a particular embodiment, the genetic construct or chimeric gene comprises:
a promoter regulatory sequence that is functional in plant cells, operably linked to
a DNA sequence which, when it is transcribed, generates an RNA molecule comprising at least a sense sequence and an antisense sequence which are at least partially complementary, said sense sequence comprising a sequence substantially identical to at least 18 contiguous nucleotides of a target gene (i.e in the meaning of the invention a saccharopine dehydrogenase gene), and said antisense sequence comprising a sequence substantially complementary to the sense sequence, and
optionally a terminator regulatory sequence.

In said embodiment, the DNA sequence according to the invention may have more particularly two aspects; in the first, it comprises two nucleotide sequences, which are sense and antisense, separated by a spacer nucleotide sequence or an intron that does not exhibit any homology with the target gene. The sequence cloned in the sense and antisense orientation is that whose expression in the pathogen it is intended to inhibit. The transcription of this DNA sequence thus gives a large single-stranded RNA corresponding to the sense/spacer-intron/antisense construct. This long RNA transcript can be detected by RT-PCR. Since the sense and antisense sequences are homologous, they will pair, and the spacer or intron which separates them plays the role of a loop for folding. A dsRNA is then obtained over all the homologous regions. The dsRNA is subsequently specifically degraded by an enzymatic complex called “DICER”. The degradation of the dsRNAs then forms siRNAs (“siRNA” in the figure), small double-stranded RNAs having a size of between 19 and 25 bases. These are then the siRNAs which, by pairing with the transcribed RNAs derived from the target gene will lead to their degradation via the RNA silencing machinery enzymatic machinery.

In the second aspect, the DNA sequence comprises two nucleotide sequences, which are sense and antisense, of different sizes, the loop structure corresponding to the part of the nucleotide sequence that does not exhibit any homology with the other nucleotide sequence. The nucleotide sequence cloned in the sense orientation is essentially homologous to the sequence of the target gene whose expression it is intended to inhibit. The antisense nucleotide sequence is essentially homologous to the complementary strand of the sequence of said target gene. The transcription of this DNA sequence thus gives a large single-stranded RNA corresponding to the sense/antisense construct. This long RNA transcript can be detected by RT-PCR. The homologous sense/antisense sequences are paired. A dsRNA is then obtained over all the homologous regions. The dsRNA is subsequently specifically degraded by an enzymatic complex called “DICER”. The degradation of the dsRNAs then forms siRNAs (“siRNA” in the figure), small doubled-stranded RNAs having a size of between 18 and 25 bases. These are then the siRNAs which, by pairing with the target RNAs, will lead to their degradation via the RNA silencing machinery plant's enzymatic machinery.

In another particular embodiment, the genetic construct comprises:
two promoter regulatory sequences that are functional in plant cells, wherein the first promoter regulatory sequence is operably linked to a DNA sequence which, when it is transcribed, generates an RNA molecule comprising at least a sense sequence, and the second promoter regulatory sequence is operably linked to a DNA sequence which, when it is transcribed, generates an RNA molecule comprising at least an antisense sequence partially complementary to the sense sequence, and wherein said sense sequence comprises a sequence substantially identical to at least 18 contiguous nucleotides of the target gene, and
optionally terminator regulatory sequence(s).

In this particular embodiment, the genetic construct may be comprised as two chimeric genes, one comprising the first promoter regulatory sequence operably linked to the first DNA sequence which, when it is transcribed, generates an RNA molecule comprising at least a sense sequence substantially identical to at least 18 contiguous nucleotides of the target gene, and optionally a terminator regulatory sequence, and the second chimeric genes comprising the second promoter regulatory sequence operably linked to the second DNA sequence which, when it is transcribed, generates an RNA molecule comprising at least an antisense sequence partially complementary to the sense sequence, and optionally a terminator regulatory sequence.

These two chimeric genes are preferably introduced into the plant cell conjointly, but not necessary, in order to favorize the hybridization of the two RNA single strands to form the dsRNA.

Alternatively, the genetic construct may be comprised as a construct comprising:
a first promoter, operably linked to
a double strand DNA sequence wherein one strand when it is transcribed under the control of the first promoter, generates an RNA molecule comprising at least a sense sequence substantially identical to at least 18 contiguous nucleotides of the target gene, and optionally a terminator regulatory sequence, and wherein the second strand, when it is transcribed under the control of the second promoter, generates an RNA molecule comprising at least an antisense sequence partially complementary to the sense sequence, and optionally a terminator regulatory sequence, and
a second promoter, in the opposite direction that the first one.

First and second promoter regulatory sequences may be different or identical, preferably different.

The invention further relates to a cloning and/or expression vector for transforming a plant, characterized in that it contains at least one chimeric gene or genetic construct as defined herein.

The present invention further relates to a transgenic plant cell containing the dsRNA molecule of the invention and as herein defined.

The present invention therefore relates to a transgenic plant cell containing the genetic construct or chimeric gene of the invention as herein defined.

In a particular embodiment of the invention, the transgenic plant cell is a soybean, oilseed, rice or potato plant cell.

The present invention further relates to a transgenic plant, seed or part thereof, comprising a transgenic plant cell according to the invention.

In a particular embodiment of the invention, the transgenic plant, seed or part thereof, is a soybean, oilseed, rice or potato plant, seed or part thereof.

According to the invention, the expression “chimeric gene” or “expression cassette” is intended to mean a nucleotide sequence comprising, functionally linked to one another in the direction of transcription, a regulatory promoter sequence that is functional in plants, a sequence encoding a protein or an RNA chain, and, optionally, a terminator that is functional in plant cells. The term “chimeric gene” or “expression cassette” is generally intended to mean a gene for which certain elements are not present in the native gene, but have been substituted for elements present in the native gene or have been added.

According to the invention, the terms “chimeric gene” or “expression cassette” may also correspond to the case where all the elements of the gene are present in the native gene, and alternatively, the term “gene” may correspond to a chimeric gene.

The expression “chimeric gene” or “expression cassette” may also correspond to the case where the sequence encoding a protein or a RNA chain is not directly linked to a promoter, but is part, for example, of a polycistronic construct comprising several coding sequences under the control of the same promoter. In that case, each coding sequences under the control of the promoter is designed as a “chimeric gene” or “expression cassette”.

According to the invention, the expression “functionally linked to one another” means that said elements of the elemental chimeric gene are linked to one another in such a way that their function is coordinated and allows the expression of the coding sequence. By way of example, a promoter is functionally linked to a coding sequence when it is capable of ensuring the expression of said coding sequence. The construction of the chimeric gene according to the invention and the assembly of its various elements can be carried out using techniques well known to those skilled in the art, in particular those described in Sambrook et al. (2001, Molecular Cloning: A Laboratory Manual (third edition), Nolan C. ed., New York: Cold Spring Harbor Laboratory Press). The choice of the regulatory elements constituting the chimeric gene depends essentially on the plant and on the type of cell in which they must function, and those skilled in the art are capable of selecting regulatory elements that are functional in a given plant.

According to the invention, the term “promoter regulatory sequence” is intended to mean any promoter regulatory sequence of a gene that is naturally expressed in plants, in particular a promoter that is expressed especially in the leaves of plants, for instance promoters referred to as constitutive of bacterial, viral or plant origin, or else promoters referred to as light-dependent, such as that of a plant ribulose-biscarboxylase/oxygenase (RuBisCO) small subunit gene, or any known suitable promoter that can be used. Among the promoters of plant origin, mention will be made of the histone promoters as described in application EP 0 507 698, or the rice actin promoter (U.S. Pat. No. 5,641,876). Among the promoters of a plant virus gene, mention will be made of that of the cauliflower mosaic virus (CaMV 19S or 35S) or of the cassava vein mosaic virus (CsVMV: WO97/48819) or the circovirus promoter (AU 689 311). Use may also be made of a promoter regulatory sequence specific for particular regions or tissues of plants, and more particularly seed-specific promoters (Datla, R. et al., 1997, Biotechnology Ann. Rev., 3, 269-296), especially the napin (EP 255 378), phaseolin, glutenin, helianthinin (WO 92/17580), albumin (WO 98/45460) and oleosin (WO 98/45461) promoters. An inducible promoter can also be used, it can be advantageously chosen from the promoters of phenylalanine ammonia lyase (PAL), of HMG-CoA reductase (HMG), of chitinases, of glucanases, of proteinase inhibitors (PI), of genes of the PR1 family, of nopaline synthase (nos) or of the vspB gene (U.S. Pat. No. 5,670,349), the HMG2 promoter (U.S. Pat. No. 5,670,349), the apple beta-galactosidase (ABG1) promoter or the apple amino cyclopropane carboxylate synthase (ACC synthase) promoter (WO 98/45445).

The term “terminator regulatory sequence” is intended to mean any sequence that is functional in plant cells or plants, also comprising polyadenylation sequences, whether they are of bacterial origin, for instance the nos or ocs terminator of Agrobacterium tumefaciens, of viral origin, for instance the CaMV 35S terminator, or else of plant origin, for instance a histone terminator as described in application EP 0 633 317.

The selection step for identifying the transformed cells and/or plants having integrated the construct according to the invention can be carried out by virtue of the presence of a selectable gene present in the construct according to the invention or in the plasmid used for the transformation of the cells or of the plants and comprising said construct. The selectable gene may be in the form of a chimeric gene comprising the following elements, functionally linked in the direction of transcription: a promoter regulatory sequence that is functional in plant cells, a sequence encoding a selectable marker, and a terminator regulatory sequence that is functional in plant cells.

Among the selectable markers that can be used, mention may be made of markers containing genes for resistance to antibiotics, such as, for example, that of the hygromycin phosphotransferase gene (Gritz et al., 1983, Gene 25: 179-188), of the neomycin phosphotransferase II gene inducing resistance to kanamycin (Wirtz et al., 1987, DNA, 6(3): 245-253), or of the aminoglycoside 3″-adenyltransferase gene, but also markers containing genes for tolerance to herbicides, such as the bar gene (White et al., NAR 18: 1062, 1990) for tolerance to bialaphos, the EPSPS gene (U.S. Pat. No. 5,188,642) for tolerance to glyphosate or else the HPPD gene (WO 96/38567) for tolerance to isoxazoles. Mention may also be made of genes encoding readily indentifiable enzymes, such as the GUS enzyme, GFP protein or genes encoding pigments or enzymes regulating pigment production in the transformed cells. Such selectable marker genes are in particular described in patent applications WO 91/02071, WO 95/06128, WO 96/38567, and WO 97/04103.

The present invention further relates to a method of making a transgenic plant cell or plant capable of expressing a dsRNA that inhibits a fungus or oomycete saccharopine dehydrogenase gene, wherein said method comprises the steps of transforming a plant cell with a chimeric gene or genetic construct according to the invention.

The method may further comprise the step of selecting the plant cell which has been transformed.

In a particular embodiment of the invention, the invention relates to a method of making a transgenic plant cell or plant capable of expressing a dsRNA that inhibits fungus or oomycete saccharopine dehydrogenase gene, wherein said method comprises the steps of transforming a plant cell with a chimeric gene or genetic construct according to the invention, and wherein said plant cell is a soybean, oilseed, rice or potato plant cell or said plant is a soybean, oilseed, rice or potato plant.

The present invention also relates to the transformed plants or part thereof, and to plants or part thereof which are derived by cultivating and/or crossing the above regenerated plants, and to the seeds of the transformed plants.

The present invention also relates to the end products such as the meal, oil, fiber which are obtained from the plants, part thereof, or seeds of the invention.

To obtain the cells or plants according to the invention, those skilled in the art can use one of the numerous known methods of transformation.

One of these methods consists in bringing the cells or tissues of the host organisms to be transformed into contact with polyethylene glycol (PEG) and the vectors of the invention (Chang and Cohen, 1979, Mol. Gen. Genet. 168(1), 111-115; Mercenier and Chassy, 1988, Biochimie 70(4), 503-517). Electroporation is another method, which consists in subjecting the cells or tissues to be transformed and the vectors of the invention to an electric field (Andreason and Evans, 1988, Biotechniques 6(7), 650-660; Shigekawa and Dower, 1989, Aust. J. Biotechnol. 3(1), 56-62). Another method consists in directly injecting the vectors into the cells or the tissues by microinjection (Gordon and Ruddle, 1985, Gene 33(2), 121-136). Advantageously, the “biolistic” method may be used. It consists in bombarding cells or tissues with particles onto which the vectors of the invention are adsorbed (Bruce et al., 1989, Proc. Natl. Acad. Sci. USA 86(24), 9692-9696; Klein et al., 1992, Biotechnology 10(3), 286-291; U.S. Pat. No. 4,945,050). Preferably, the transformation of plant cells or tissues can be carried out using bacteria of the Agrobacterium genus, preferably by infection of the cells or tissues of said plants with A. tumefaciens (Knopf, 1979, Subcell. Biochem. 6, 143-173; Shaw et al., 1983, Gene 23(3): 315-330) or A. rhizogenes (Bevan and Chilton, 1982, Annu. Rev. Genet. 16: 357-384; Tepfer and Casse-Delbart, 1987, Microbiol. Sci. 4(1), 24-28). Preferably, the transformation of plant cells or tissues with Agrobacterium tumefaciens is carried out according to the protocol described by Hiei et al., (1994, Plant J. 6(2): 271-282). Those skilled in the art will choose the appropriate method according to the nature of the host organisms to be transformed.

The plants according to the invention contain transformed plant cells as defined above. In particular, the transformed plants can be obtained by regeneration of the transformed plant cells described above. The regeneration is obtained by any appropriate method, which depends on the nature of the species.

The invention also comprises parts of these plants, and the progeny of these plants. The term “part of these plants” is intended to mean any organ of these plants, whether above ground or below ground. The organs above ground are the stems, the leaves and the flowers comprising the male and female reproductive organs. The organs below ground are mainly the roots, but they may also be tubers. The term “progeny” is intended to mean mainly the seeds containing the embryos derived from the reproduction of these plants with one another. By extension, the term “progeny” applies to all the seeds formed at each new generation derived from crosses between the transformed plants according to the invention. Progeny and seeds can also be obtained by vegetative multiplication of said transformed plants. The seeds according to the invention can be coated with an agrochemical composition comprising at least one active product having an activity selected from fungicidal, herbicidal, insecticidal, nematicidal, bactericidal or virucidal activities.

The invention further relates to a method for controlling a plant pathogen, particularly a fungus or an oomycete, comprising providing to said pathogen a dsRNA molecule according to the invention and as herein defined.

In a particular embodiment of the invention, the method relates to a method for controlling a plant pathogen, particularly a fungus or an oomycete, comprising providing to said pathogen a dsRNA according to the invention and as herein defined, or a composition comprising said dsRNA, wherein said plant pathogen is Magnaporthe grisea, Phytophthora infestans, Sclerotinia sclerotiorum or Phakopsora pachyrhizi.

In a particular embodiment of the invention, the method relates to a method for controlling a plant pathogen, particularly a fungus or an oomycete, comprising providing to said pathogen a dsRNA molecule according to the invention and as herein defined, or a composition comprising said dsRNA, wherein said plant is a soybean, oilseed, rice or potato plant.

The invention further relates to a method for controlling a plant, crop or seed pathogen, particularly a fungus or an oomycete, characterized in that an agronomically effective and substantially non-phytotoxic quantity of dsRNA molecule according to the invention or composition according to the invention is applied as seed treatment, foliar application, stem application, drench or drip application (chemigation) to the seed, the plant or to the fruit of the plant or to soil or to inert substrate (e.g. inorganic substrates like sand, rockwool, glasswool; expanded minerals like perlite, vermiculite, zeolite or expanded clay), Pumice, Pyroclastic materials or stuff, synthetic organic substrates (e.g. polyurethane) organic substrates (e.g. peat, composts, tree waste products like coir, wood fibre or chips, tree bark) or to a liquid substrate (e.g. floating hydroponic systems, Nutrient Film Technique, Aeroponics) wherein the plant is growing or wherein it is desired to grow.

The invention therefore relates to a method for controlling a plant pathogen, particularly a fungus or oomycete, characterized in that an effective quantity of a dsRNA molecule according to the invention or a composition according to the invention is applied to the soil where plants grow or are capable of growing, to the leaves and/or the fruit of plants or to the seeds of plants.

In a particular embodiment of the invention, the invention relates to a method for controlling a plant pathogen, particularly a fungus or oomycete, characterized in that an effective quantity of a dsRNA molecule according to the invention or a composition according to the invention is applied to the soil where plants grow or are capable of growing, to the leaves and/or the fruit of plants or to the seeds of plants, wherein said plant pathogen is Magnaporthe grisea, Phytophthora infestans, Sclerotinia sclerotinium or Phakopsora pachyrhizi.

In a particular embodiment of the invention, the invention relates to a method for controlling a plant pathogen, particularly a fungus or oomycete, characterized in that an effective quantity of a dsRNA molecule according to the invention or a composition according to the invention is applied to the soil where plants grow or are capable of growing, to the leaves and/or the fruit of plants or to the seeds of plants, wherein said plant is a soybean, oilseed, rice or potato plant.

The expression “are applied to the plants to be treated” is understood to mean, for the purposes of the present invention, that the pesticide composition which is the subject of the invention can be applied by means of various methods of treatment such as:
spraying onto the aerial parts of the said plants a liquid comprising one of the said compositions,
dusting, the incorporation into the soil of granules or powders, spraying, around the said plants and in the case of trees injection or daubing,
coating or film-coating the seeds of the said plants with the aid of a plant-protection mixture comprising one of the said compositions.

The method according to the invention can either be a curing, preventing or eradicating method.

In this method, a composition used can be prepared beforehand by mixing the two or more active compounds according to the invention.

According to an alternative of such a method, it is also possible to apply simultaneously, successively or separately compounds (A) and (B) so as to have the conjugated (A)/(B) effects, of distinct compositions each containing one of the two or three active ingredients (A) or (B).

The dose of active dsRNA compound usually applied in the method of treatment according to the invention is generally and advantageously
for foliar treatments: from 0.0001 to 10,000 g/ha, preferably from 0,0001 to 1000 g/ha, more preferably from 0.001 to 300 g/ha; in case of drench or drip application, the dose can even be reduced, especially while using inert substrates like rockwool or perlite;
for seed treatment: from 0.0001 to 200 g per 100 kilogram of seed, preferably from 0.001 to 150 g per 100 kilogram of seed;
for soil treatment: from 0.0001 to 10,000 g/ha, preferably from 0.001 to 5,000 g/ha.

When the dsRNA of the invention is mixed with another active phytopharmaceutical or plant growth promoting compound, said phytopharmaceutical or plant growth promoting compound is used in the dose usually applied.

Said phytopharmaceutical or plant growth promoting compound may be a fungicide, herbicide, insecticide, nematicide, acaricide, molluscicide, resistance inducer, safeners, or signal compounds. The dose of phytopharmaceutical active compound usually applied in the method of treatment according to the invention is generally and advantageously from 10 to 800 g/ha, preferably from 50 to 300 g/ha for applications in foliar treatment. The dose of active substance applied is generally and advantageously from 2 to 200 g per 100 kg of seed, preferably from 3 to 150 g per 100 kg of seed in the case of seed treatment.

The doses herein indicated are given as illustrative Examples of method according to the invention. A person skilled in the art will know how to adapt the application doses, notably according to the nature of the plant or crop to be treated.

Under specific conditions, for example according to the nature of the pathogen, phytopathogenic fungus or oomycete to be treated or controlled, a lower dose can offer adequate protection. Certain climatic conditions, resistance or other factors like the nature of the pathogen or the degree of infestation, for example, of the plants with these pathogens, can require higher doses of combined active ingredients. The optimum dose usually depends on several factors, for example on the type of pathogen to be treated, on the type or level of development of the infested plant or plant material, on the density of vegetation or alternatively on the method of application.

Without it being limiting, the crop treated with the pesticide composition or combination according to the invention is, for example, grapevine, cereals, vegetables, lucerne, soybean, market garden crops, turf, wood, tree or horticultural plants.

The method of treatment according to the invention can also be useful to treat propagation material such as tubers or rhizomes, but also seeds, seedlings or seedlings pricking out and plants or plants pricking out. This method of treatment can also be useful to treat roots. The method of treatment according to the invention can also be useful to treat the over-ground parts of the plant such as trunks, stems or stalks, leaves, flowers and fruit of the concerned plant, and in general every material which is susceptiblel to fungal infection (e.g due to storage like hay)

The invention further relates to a method of controlling a plant pathogen, particularly a fungus or an oomycete, comprising providing in the host plant of said plant pathogen a transformed plant cell according to the invention.

The invention further relates to a method of controlling a plant pathogen, particularly a fungus or an oomycete, comprising providing in the host plant of said plant pathogen a transformed plant cell containing a dsRNA as herein defined.

The invention further relates to a method of controlling a plant pathogen, particularly a fungus or an oomycete, comprising transforming the plant with a genetic construct according to the invention.

The invention further relates to a method of controlling a plant pathogen, particularly a fungus or an oomycete, comprising the following steps:

i) transforming a plant cell with a chimeric gene according to the invention;
ii) placing the cells thus transformed under conditions that allow the transcription of said construct,
iii) having the cells into contact with the pathogen.

In a particular embodiment of the invention, methods according to the invention are controlling a plant pathogen selected from Magnaporthe grisea, Phytophthora infestans, Sclerotinia sclerotinium or Phakopsora pachyrhizi.

In a particular embodiment of the invention, methods according to the invention are controlling a plant pathogen wherein the plant is a soybean, oilseed, rice or potato plant.

The invention further relates to a method for inhibiting the expression of a plant pathogen, particularly fungus or oomycete, saccharopine dehydrogenase gene, comprising the following steps:

i) transforming a plant cell with a chimeric gene according to the invention;
ii) placing the cells thus transformed under conditions that allow the transcription of said construct,
iii) having the cells into contact with the pathogen.

In a particular embodiment of the invention, the method according to the invention is inhibiting a fungal or oomycete saccharopine dehydrogenase gene, wherein the fungus or oomycete is Magnaporthe grisea, Phytophthora infestans, Sclerotinia sclerotinium or Phakopsora pachyrhizi.

In a particular embodiment of the invention, the method according to the invention inhibits a fungal or oomycete saccharopine dehydrogenase gene, said method comprises the following steps:

i) transforming a plant cell with a chimeric gene according to the invention;
ii) placing the cells thus transformed under conditions that allow the transcription of said construct,
iii) having the cells into contact with the pathogen;
wherein the plant is a soybean, oilseed, rice or potato plant.

According to the invention all plants and plant parts can be treated. By plants is meant all plants and plant populations such as desirable and undesirable wild plants, cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed mutagenesis, molecular or genetic markers or by bioengineering and genetic engineering methods. By plant parts is meant all above ground and below ground parts and organs of plants such as shoot, leaf, blossom and root, whereby for example leaves, needles, stems, branches, blossoms, fruiting bodies, fruits and seed as well as roots, corms and rhizomes are listed. Crops and vegetative and generative propagating material, for example cuttings, corms, rhizomes, runners and seeds also belong to plant parts.

Among the plants that can be protected by the method according to the invention, mention may be made of major field crops like corn, soybean, cotton, Brassica oilseeds such as Brassica napus (e.g. canola), Brassica rapa, B. juncea (e.g. mustard) and Brassica carinata, rice, wheat, sugarbeet, sugarcane, oats, rye, barley, millet, triticale, flax, vine and various fruits and vegetables of various botanical taxa such as Rosaceae sp. (for instance pip fruit such as apples and pears, but also stone fruit such as apricots, cherries, almonds and peaches, berry fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for instance banana trees and plantings), Rubiaceae sp. (for instance coffee), Theaceae sp., Sterculiceae sp., Rutaceae sp. (for instance lemons, oranges and grapefruit); Solanaceae sp. (for instance tomatoes, potatoes, peppers, eggplant), Liliaceae sp., Compositiae sp. (for instance lettuce, artichoke and chicory—including root chicory, endive or common chicory), Umbeffiferae sp. (for instance carrot, parsley, celery and celeriac), Cucurbitaceae sp. (for instance cucumber—including pickling cucumber, squash, watermelon, gourds and melons), Alliaceae sp. (for instance onions and leek), Cruciferae sp. (for instance white cabbage, red cabbage, broccoli, cauliflower, brussel sprouts, pak choi, kohlrabi, radish, horseradish, cress, Chinese cabbage), Leguminosae sp. (for instance peanuts, peas and beans beans—such as climbing beans and broad beans), Chenopodiaceae sp. (for instance mangold, spinach beet, spinach, beetroots), Malvaceae (for instance okra), Asparagaceae (for instance asparagus); horticultural and forest crops; ornamental plants; as well as genetically modified homologues of these crops.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology or RNA interference—RNAi-technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates, the active compound combinations according to the invention may also have a strengthening effect in plants. Accordingly, they are also suitable for mobilizing the defense system of the plant against attack by unwanted microorganisms. This may, if appropriate, be one of the reasons of the enhanced activity of the combinations according to the invention, for example against fungi. Plant-strengthening (resistance-inducing) substances are to be understood as meaning, in the present context, those substances or combinations of substances which are capable of stimulating the defense system of plants in such a way that, when subsequently inoculated with unwanted microorganisms, the treated plants display a substantial degree of resistance to these microorganisms. In the present case, unwanted microorganisms are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

As already mentioned above, it is possible to treat all plants and their parts in accordance with the invention. In a preferred embodiment, wild plant species and plant cultivars, or those obtained by conventional biological breeding methods, such as crossing or protoplast fusion, and also parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts” or “parts of plants” or “plant parts” have been explained above. More preferably, plants of the plant cultivars which are commercially available or are in use are treated in accordance with the invention. Plant cultivars are understood to mean plants which have new properties (“traits”) and have been obtained by conventional breeding, by mutagenesis or by recombinant DNA techniques. They can be cultivars, varieties, bio- or genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi-technology or microRNA—miRNA-technology). A heterologous gene that is located in the genome is also called a transgene. A transgene that is defined by its particular location in the plant genome is called a transformation or transgenic event.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf color, earlier flowering, higher quality and/or a higher nutritional value of the harvested products, higher sugar concentration within the fruits, better storage stability and/or processability of the harvested products are possible, which exceed the effects which were actually to be expected.

At certain application rates, the active compound combinations according to the invention may also have a strengthening effect in plants. Accordingly, they are also suitable for mobilizing the defense system of the plant against attack by unwanted microorganisms. This may, if appropriate, be one of the reasons of the enhanced activity of the combinations according to the invention, for example against fungi. Plant-strengthening (resistance-inducing) substances are to be understood as meaning, in the present context, those substances or combinations of substances which are capable of stimulating the defense system of plants in such a way that, when subsequently inoculated with unwanted microorganisms, the treated plants display a substantial degree of resistance to these microorganisms. In the present case, unwanted microorganisms are to be understood as meaning phytopathogenic fungi, bacteria and viruses. Thus, the substances according to the invention can be employed for protecting plants against attack by the abovementioned pathogens within a certain period of time after the treatment. The period of time within which protection is effected generally extends from 1 to 10 days, preferably 1 to 7 days, after the treatment of the plants with the active compounds.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Examples of nematode or insect resistant plants are described in e.g. U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396, 12/497,221, 12/644,632, 12/646,004, 12/701,058, 12/718,059, 12/721,595, 12/638,591.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses). Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e. the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species (WO 92/05251, WO 95/09910, WO 98/27806, WO 05/002324, WO 06/021972 and U.S. Pat. No. 6,229,072). However, genetic determinants for male sterility can also be located in the nuclear genome. Male sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar (e.g. WO 91/02069).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-resistant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Science 1983, 221, 370-371), the CP4 gene of the bacterium Agrobacterium sp. (Curr. Topics Plant Physiol. 1992, 7, 139-145), the genes encoding a Petunia EPSPS (Science 1986, 233, 478-481), a Tomato EPSPS (J. Biol. Chem. 1988, 263, 4280-4289), or an Eleusine EPSPS (WO 01/66704). It can also be a mutated EPSPS as described in for example EP 0837944, WO 00/66746, WO 00/66747 or WO 02/26995. Glyphosate-tolerant plants can also be obtained by expressing a gene that encodes a glyphosate oxido-reductase enzyme as described in U.S. Pat. Nos. 5,776,760 and 5,463,175. Glyphosate-tolerant plants can also be obtained by expressing a gene that encodes a glyphosate acetyl transferase enzyme as described in for example WO 02/036782, WO 03/092360, WO 2005/012515 and WO 2007/024782. Glyphosate-tolerant plants can also be obtained by selecting plants containing naturally-occurring mutations of the above-mentioned genes, as described in for example WO 01/024615 or WO 03/013226. Plants expressing EPSPS genes that confer glyphosate tolerance are described in e.g. U.S. patent application Ser. Nos. 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943,801 or 12/362,774. Plants comprising other genes that confer glyphosate tolerance, such as decarboxylase genes, are described in e.g. U.S. patent application Ser. Nos. 11/588,811, 11/185,342, 12/364,724, 11/185,560 or 12/423,926.

Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Plants expressing an exogenous phosphinothricin acetyltransferase are for example described in U.S. Pat. Nos. 5,561,236; 5,648,477; 5,646,024; 5,273,894; 5,637,489; 5,276,268; 5,739,082; 5,908,810 and 7,112,665.

Further herbicide-tolerant plants are also plants that are made tolerant to the herbicides inhibiting the enzyme hydroxyphenylpyruvatedioxygenase (HPPD). HPPD is an enzyme that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, WO 99/24586, WO 09/144079, WO 02/046387, or U.S. Pat. No. 6,768,044. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate deshydrogenase (PDH) activity in addition to a gene encoding an HPPD-tolerant enzyme, as described in WO 04/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 2007/103567 and WO 2008/150473.

Still further herbicide resistant plants are plants that are made tolerant to acetolactate synthase (ALS) inhibitors. Known ALS-inhibitors include, for example, sulfonylurea, imidazolinone, triazolopyrimidines, pryimidinyoxy(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright (Weed Science 2002, 50, 700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and WO 96/33270. Other imidazolinone-tolerant plants are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerant plants are also described in for example WO 2007/024782 and U.S. Patent Application 61/288,958.

Other plants tolerant to imidazolinone and/or sulfonylurea can be obtained by induced mutagenesis, selection in cell cultures in the presence of the herbicide or mutation breeding as described for example for soybeans in U.S. Pat. No. 5,084,082, for rice in WO 97/41218, for sugar beet in U.S. Pat. No. 5,773,702 and WO 99/057965, for lettuce in U.S. Pat. No. 5,198,599, or for sunflower in WO 01/065922.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:
1) an insecticidal crystal protein from Bacillus thuringiensis or an insecticidal portion thereof, such as the insecticidal crystal proteins listed by Crickmore et al. (1998, Microbiology and Molecular Biology Reviews, 62: 807-813), updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP-A 1 999 141 and WO 2007/107302), or such proteins encoded by synthetic genes as e.g. described in and U.S. patent application Ser. No. 12/249,016; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Nat. Biotechnol. 2001, 19, 668-72; Applied Environm. Microbiol. 2006, 71, 1765-1774) or the binary toxin made up of the Cry1A or Cry1F proteins and the Cry2Aa or Cry2Ab or Cry2Ae proteins (U.S. patent application Ser. No. 12/214,022 and EP-A 2 300 618); or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 2007/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or
9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Applications 61/126,083 and 61/195,019), or the binary toxin made up of the VIP3 protein and the Cry2Aa or Cry2Ab or Cry2Ae proteins (U.S. patent application Ser. No. 12/214,022 and EP-A 2 300 618).
10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein)

Of course, an insect-resistant transgenic plant, as used herein, also includes any plant comprising a combination of genes encoding the proteins of any one of the above classes 1 to 10. In one embodiment, an insect-resistant plant contains more than one transgene encoding a protein of any one of the above classes 1 to 10, to expand the range of target insect species affected when using different proteins directed at different target insect species, or to delay insect resistance development to the plants by using different proteins insecticidal to the same target insect species but having a different mode of action, such as binding to different receptor binding sites in the insect.

An “insect-resistant transgenic plant”, as used herein, further includes any plant containing at least one transgene comprising a sequence producing upon expression a double-stranded RNA which upon ingestion by a plant insect pest inhibits the growth of this insect pest, as described e.g. in WO 2007/080126, WO 2006/129204, WO 2007/074405, WO 2007/080127 and WO 2007/035650. Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:
1) plants which contain a transgene capable of reducing the expression and/or the activity of poly(ADP-ribose) polymerase (PARP) gene in the plant cells or plants as described in WO 00/04173, WO 2006/045633, EP-A 1 807 519, or EP-A 2 018 431.
2) plants which contain a stress tolerance enhancing transgene capable of reducing the expression and/or the activity of the PARC encoding genes of the plants or plants cells, as described e.g. in WO 2004/090140.
3) plants which contain a stress tolerance enhancing transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP-A 1 794 306, WO 2006/133827, WO 2007/107326, EP-A 1 999 263, or WO 2007/107326.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as:
1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or plants, so that this is better suited for special applications. Said transgenic plants synthesizing a modified starch are disclosed, for example, in EP-A 0 571 427, WO 95/04826, EP-A 0 719 338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO 99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 04/056999, WO 05/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 00/22140, WO 2006/063862, WO 2006/072603, WO 02/034923, WO 2008/017518, WO 2008/080630, WO 2008/080631, EP 07090007.1, WO 2008/090008, WO 01/14569, WO 02/79410, WO 03/33540, WO 2004/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 2005/002359, U.S. Pat. Nos. 5,824,790, 6,013,861, WO 94/04693, WO 94/09144, WO 94/11520, WO 95/35026, WO 97/20936, WO 2010/012796, WO 2010/003701,
2) transgenic plants which synthesize non starch carbohydrate polymers or which synthesize non starch carbohydrate polymers with altered properties in comparison to wild type plants without genetic modification. Examples are plants producing polyfructose, especially of the inulin and levan-type, as disclosed in EP-A 0 663 956, WO 96/01904, WO 96/21023, WO 98/39460, and WO 99/24593, plants producing alpha-1,4-glucans as disclosed in WO 95/31553, US 2002031826, U.S. Pat. Nos. 6,284,479, 5,712,107, WO 97/47806, WO 97/47807, WO 97/47808 and WO 00/14249, plants producing alpha-1,6 branched alpha-1,4-glucans, as disclosed in WO 00/73422, plants producing alternan, as disclosed in e.g. WO 00/47727, WO 00/73422, EP 06077301.7, U.S. Pat. No. 5,908,975 and EP-A 0 728 213,
3) transgenic plants which produce hyaluronan, as for example disclosed in WO 2006/032538, WO 2007/039314, WO 2007/039315, WO 2007/039316, JP-A 2006-304779, and WO 2005/012529.
4) transgenic plants or hybrid plants, such as onions with characteristics such as ‘high soluble solids content’, ‘low pungency’ (LP) and/or ‘long storage’ (LS), as described in U.S. patent application Ser. No. 12/020,360 and 61/054,026.

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:
a) Plants, such as cotton plants, containing an altered form of cellulose synthase genes as described in WO 98/00549.
b) Plants, such as cotton plants, containing an altered form of rsw2 or rsw3 homologous nucleic acids as described in WO 2004/053219.
c) Plants, such as cotton plants, with increased expression of sucrose phosphate synthase as described in WO 01/17333.
d) Plants, such as cotton plants, with increased expression of sucrose synthase as described in WO 02/45485.
e) Plants, such as cotton plants, wherein the timing of the plasmodesmatal gating at the basis of the fiber cell is altered, e.g. through downregulation of fiber-selective β-1,3-glucanase as described in WO 2005/017157, or as described in WO 2009/143995.
f) Plants, such as cotton plants, having fibers with altered reactivity, e.g. through the expression of N-acetylglucosaminetransferase gene including nodC and chitin synthase genes as described in WO 2006/136351.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics and include:
a) Plants, such as oilseed rape plants, producing oil having a high oleic acid content as described e.g. in U.S. Pat. Nos. 5,969,169, 5,840,946 or 6,323,392 or 6,063,947
b) Plants such as oilseed rape plants, producing oil having a low linolenic acid content as described in U.S. Pat. Nos. 6,270,828, 6,169,190, or 5,965,755
c) Plant such as oilseed rape plants, producing oil having a low level of saturated fatty acids as described e.g. in U.S. Pat. No. 5,434,283 or U.S. patent application Ser. No. 12/668,303

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering as described in U.S. Patent Application 61/135,230, WO 2009/068313 and WO 2010/006732.

Plants or plant cultivars (that can be obtained by plant biotechnology methods such as genetic engineering) which may also be treated according to the invention are plants, such as Tobacco plants, with altered post-translational protein modification patterns, for example as described in WO 2010/121818 and WO 2010/145846.

Particularly useful transgenic plants which may be treated according to the invention are plants containing transformation events, or combination of transformation events, that are the subject of petitions for non-regulated status, in the United States of America, to the Animal and Plant Health Inspection Service (APHIS) of the United States Department of Agriculture (USDA) whether such petitions are granted or are still pending. At any time this information is readily available from APHIS (4700 River Road, Riverdale, Md. 20737, USA), for instance on its internet site (URL http://www.aphis.usda.gov/brs/not_reg.html). On the filing date of this application the petitions for nonregulated status that were pending with APHIS or granted by APHIS were those which contains the following information:
Petition: the identification number of the petition. Technical descriptions of the transformation events can be found in the individual petition documents which are obtainable from APHIS, for example on the APHIS website, by reference to this petition number. These descriptions are herein incorporated by reference.
Extension of Petition: reference to a previous petition for which an extension is requested.
Institution: the name of the entity submitting the petition.
Regulated article: the plant species concerned.
Transgenic phenotype: the trait conferred to the plants by the transformation event.
Transformation event or line: the name of the event or events (sometimes also designated as lines or lines) for which nonregulated status is requested.
APHIS documents: various documents published by APHIS in relation to the Petition and which can be requested with APHIS.

Additional particularly useful plants containing single transformation events or combinations of transformation events are listed for example in the databases from various national or regional regulatory agencies (see for example http://gmoinfo.jrc.it/gmp_browse.aspx and http://www.agbios.com/dbase.php).

Particularly useful transgenic plants which may be treated according to the invention are plants containing transformation events, or a combination of transformation events, and that are listed for example in the databases for various national or regional regulatory agencies including Event 1143-14A (cotton, insect control, not deposited, described in WO 2006/128569); Event 1143-51B (cotton, insect control, not deposited, described in WO 2006/128570); Event 1445 (cotton, herbicide tolerance, not deposited, described in US-A 2002-120964 or WO 02/034946); Event 17053 (rice, herbicide tolerance, deposited as PTA-9843, described in WO 2010/117737); Event 17314 (rice, herbicide tolerance, deposited as PTA-9844, described in WO 2010/117735); Event 281-24-236 (cotton, insect control-herbicide tolerance, deposited as PTA-6233, described in WO 2005/103266 or US-A 2005-216969); Event 3006-210-23 (cotton, insect control-herbicide tolerance, deposited as PTA-6233, described in US-A 2007-143876 or WO 2005/103266); Event 3272 (corn, quality trait, deposited as PTA-9972, described in WO 2006/098952 or US-A 2006-230473); Event 40416 (corn, insect control-herbicide tolerance, deposited as ATCC PTA-11508, described in WO 2011/075593); Event 43A47 (corn, insect control-herbicide tolerance, deposited as ATCC PTA-11509, described in WO 2011/075595); Event 5307 (corn, insect control, deposited as ATCC PTA-9561, described in WO 2010/077816); Event ASR-368 (bent grass, herbicide tolerance, deposited as ATCC PTA-4816, described in US-A 2006-162007 or WO 2004/053062); Event B16 (corn, herbicide tolerance, not deposited, described in US-A 2003-126634); Event BPS-CV127-9 (soybean, herbicide tolerance, deposited as NCIMB No. 41603, described in WO 2010/080829); Event CE43-67B (cotton, insect control, deposited as DSM ACC2724, described in US-A 2009-217423 or WO2006/128573); Event CE44-69D (cotton, insect control, not deposited, described in US-A 2010-0024077); Event CE44-69D (cotton, insect control, not deposited, described in WO 2006/128571); Event CE46-02A (cotton, insect control, not deposited, described in WO 2006/128572); Event COT102 (cotton, insect control, not deposited, described in US-A 2006-130175 or WO 2004/039986); Event COT202 (cotton, insect control, not deposited, described in US-A 2007-067868 or WO 2005/054479); Event COT203 (cotton, insect control, not deposited, described in WO 2005/054480); Event DAS40278 (corn, herbicide tolerance, deposited as ATCC PTA-10244, described in WO 2011/022469); Event DAS-59122-7 (corn, insect control-herbicide tolerance, deposited as ATCC PTA 11384, described in US-A 2006-070139); Event DAS-59132 (corn, insect control-herbicide tolerance, not deposited, described in WO 2009/100188); Event DAS68416 (soybean, herbicide tolerance, deposited as ATCC PTA-10442, described in WO 2011/066384 or WO 2011/066360); Event DP-098140-6 (corn, herbicide tolerance, deposited as ATCC PTA-8296, described in US-A 2009-137395 or WO 2008/112019); Event DP-305423-1 (soybean, quality trait, not deposited, described in US-A 2008-312082 or WO 2008/054747); Event DP-32138-1 (corn, hybridization system, deposited as ATCC PTA-9158, described in US-A 2009-0210970 or WO 2009/103049); Event DP-356043-5 (soybean, herbicide tolerance, deposited as ATCC PTA-8287, described in US-A 2010-0184079 or WO 2008/002872); Event EE-1 (brinjal, insect control, not deposited, described in WO 2007/091277); Event F1117 (corn, herbicide tolerance, deposited as ATCC 209031, described in US-A 2006-059581 or WO 98/044140); Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO 98/044140); Event GHB119 (cotton, insect control-herbicide tolerance, deposited as ATCC PTA-8398, described in WO 2008/151780); Event GHB614 (cotton, herbicide tolerance, deposited as ATCC PTA-6878, described in US-A 2010-050282 or WO 2007/017186); Event GJ11 (corn, herbicide tolerance, deposited as ATCC 209030, described in US-A 2005-188434 or WO 98/044140); Event GM RZ13 (sugar beet, virus resistance, deposited as NCIMB-41601, described in WO 2010/076212); Event H7-1 (sugar beet, herbicide tolerance, deposited as NCIMB 41158 or NCIMB 41159, described in US-A 2004-172669 or WO 2004/074492); Event JOPLINI (wheat, disease tolerance, not deposited, described in US-A 2008-064032); Event LL27 (soybean, herbicide tolerance, deposited as NCIMB41658, described in WO 2006/108674 or US-A 2008-320616); Event LL55 (soybean, herbicide tolerance, deposited as NCIMB 41660, described in WO 2006/108675 or US-A 2008-196127); Event LLcotton25 (cotton, herbicide tolerance, deposited as ATCC PTA-3343, described in WO 03/013224 or US-A 2003-097687); Event LLRICE06 (rice, herbicide tolerance, deposited as ATCC-23352, described in U.S. Pat. No. 6,468,747 or WO 00/026345); Event LLRICE601 (rice, herbicide tolerance, deposited as ATCC PTA-2600, described in US-A 2008-2289060 or WO 00/026356); Event LY038 (corn, quality trait, deposited as ATCC PTA-5623, described in US-A 2007-028322 or WO 2005/061720); Event MIR162 (corn, insect control, deposited as PTA-8166, described in US-A 2009-300784 or WO 2007/142840); Event MIR604 (corn, insect control, not deposited, described in US-A 2008-167456 or WO 2005/103301); Event MON15985 (cotton, insect control, deposited as ATCC PTA-2516, described in US-A 2004-250317 or WO 02/100163); Event MON810 (corn, insect control, not deposited, described in US-A 2002-102582); Event MON863 (corn, insect control, deposited as ATCC PTA-2605, described in WO 2004/011601 or US-A 2006-095986); Event MON87427 (corn, pollination control, deposited as ATCC PTA-7899, described in WO 2011/062904); Event MON87460 (corn, stress tolerance, deposited as ATCC PTA-8910, described in WO 2009/111263 or US-A 2011-0138504); Event MON87701 (soybean, insect control, deposited as ATCC PTA-8194, described in US-A 2009-130071 or WO 2009/064652); Event MON87705 (soybean, quality trait-herbicide tolerance, deposited as ATCC PTA-9241, described in US-A 2010-0080887 or WO 2010/037016); Event MON87708 (soybean, herbicide tolerance, deposited as ATCC PTA9670, described in WO 2011/034704); Event MON87754 (soybean, quality trait, deposited as ATCC PTA-9385, described in WO 2010/024976); Event MON87769 (soybean, quality trait, deposited as ATCC PTA-8911, described in US-A 2011-0067141 or WO 2009/102873); Event MON88017 (corn, insect control-herbicide tolerance, deposited as ATCC PTA-5582, described in US-A 2008-028482 or WO 2005/059103); Event MON88913 (cotton, herbicide tolerance, deposited as ATCC PTA-4854, described in WO 2004/072235 or US-A 2006-059590); Event MON89034 (corn, insect control, deposited as ATCC PTA-7455, described in WO 2007/140256 or US-A 2008-260932); Event MON89788 (soybean, herbicide tolerance, deposited as ATCC PTA-6708, described in US-A 2006-282915 or WO 2006/130436); Event MS11 (oilseed rape, pollination control-herbicide tolerance, deposited as ATCC PTA-850 or PTA-2485, described in WO 01/031042); Event MS8 (oilseed rape, pollination control-herbicide tolerance, deposited as ATCC PTA-730, described in WO 01/041558 or US-A 2003-188347); Event NK603 (corn, herbicide tolerance, deposited as ATCC PTA-2478, described in US-A 2007-292854); Event PE-7 (rice, insect control, not deposited, described in WO 2008/114282); Event RF3 (oilseed rape, pollination control-herbicide tolerance, deposited as ATCC PTA-730, described in WO 01/041558 or US-A 2003-188347); Event RT73 (oilseed rape, herbicide tolerance, not deposited, described in WO 02/036831 or US-A 2008-070260); Event T227-1 (sugar beet, herbicide tolerance, not deposited, described in WO 02/44407 or US-A 2009-265817); Event T25 (corn, herbicide tolerance, not deposited, described in US-A 2001-029014 or WO 01/051654); Event T304-40 (cotton, insect control-herbicide tolerance, deposited as ATCC PTA-8171, described in US-A 2010-077501 or WO 2008/122406); Event T342-142 (cotton, insect control, not deposited, described in WO 2006/128568); Event TC1507 (corn, insect control-herbicide tolerance, not deposited, described in US-A 2005-039226 or WO 2004/099447); Event VIP1034 (corn, insect control-herbicide tolerance, deposited as ATCC PTA-3925, described in WO 03/052073), Event 32316 (corn, insect control-herbicide tolerance, deposited as PTA-11507, described in WO 2011/084632), Event 4114 (corn, insect control-herbicide tolerance, deposited as PTA-11506, described in WO 2011/084621).

The composition according to the invention can also be used against fungal diseases liable to grow on or inside timber. The term “timber” means all types of species of wood and all types of working of this wood intended for construction, for example solid wood, high-density wood, laminated wood and plywood. The method for treating timber according to the invention mainly consists in contacting one or more compounds according to the invention or a composition according to the invention; this includes for example direct application, spraying, dipping, injection or any other suitable means.

Among the diseases of plants or crops that can be controlled by the methods according to the invention, mention can be made of:

Powdery mildew diseases such as:
Blumeria diseases, caused for example by Blumeria graminis;
Podosphaera diseases, caused for example by Podosphaera leucotricha;
Sphaerotheca diseases, caused for example by Sphaerotheca fuliginea;
Uncinula diseases, caused for example by Uncinula necator;
   Rust diseases such as:
Gymnosporangium diseases, caused for example by Gymnosporangium sabinae;
Hemileia diseases, caused for example by Hemileia vastatrix;
Phakopsora diseases, caused for example by Phakopsora pachyrhizi or Phakopsora meibomiae;
Puccinia diseases, caused for example by Puccinia recondite, Puccinia graminis or Puccinia striiformis;
Uromyces diseases, caused for example by Uromyces appendiculatus;
   Oomycete diseases such as:
Albugo diseases caused for example by Albugo candida;
Bremia diseases, caused for example by Bremia lactucae;
Peronospora diseases, caused for example by Peronospora pisi or P. brassicae;
Phytophthora diseases, caused for example by Phytophthora infestans;
Plasmopara diseases, caused for example by Plasmopara viticola;
Pseudoperonospora diseases, caused for example by Pseudoperonospora humuli or Pseudoperonospora cubensis;
Pythium diseases, caused for example by Pythium ultimum;
   Leafspot, leaf blotch and leaf blight diseases such as:
Alternaria diseases, caused for example by Alternaria solani;
Cercospora diseases, caused for example by Cercospora beticola;
Cladiosporum diseases, caused for example by Cladiosporium cucumerinum;
Cochliobolus diseases, caused for example by Cochliobolus sativus (Conidiaform: Drechslera, Syn: Helminthosporium) or Cochliobolus miyabeanus;
Colletotrichum diseases, caused for example by Colletotrichum lindemuthanium;
Cycloconium diseases, caused for example by Cycloconium oleaginum;
Diaporthe diseases, caused for example by Diaporthe citri;
Elsinoe diseases, caused for example by Elsinoe fawcettii;
Gloeosporium diseases, caused for example by Gloeosporium laeticolor;
Glomerella diseases, caused for example by Glomerella cingulata;
Guignardia diseases, caused for example by Guignardia bidwelli;
Leptosphaeria diseases, caused for example by Leptosphaeria maculans; Leptosphaeria nodorum;
Magnaporthe diseases, caused for example by Magnaporthe grisea;
Mycosphaerella diseases, caused for example by Mycosphaerella graminicola; Mycosphaerella arachidicola; Mycosphaerella fijiensis;
Phaeosphaeria diseases, caused for example by Phaeosphaeria nodorum;
Pyrenophora diseases, caused for example by Pyrenophora teres, or Pyrenophora tritici repentis;
Ramularia diseases, caused for example by Ramularia collo-cygni, or Ramularia areola;
Rhynchosporium diseases, caused for example by Rhynchosporium secalis;
Septoria diseases, caused for example by Septoria apii or Septoria lycopercisi;
Typhula diseases, caused for example by Typhula incamata;
Venturia diseases, caused for example by Venturia inaequalis;
   Root, Sheath and stem diseases such as:
Corticium diseases, caused for example by Corticium graminearum;
Fusarium diseases, caused for example by Fusarium oxysporum;
Gaeumannomyces diseases, caused for example by Gaeumannomyces graminis;
Rhizoctonia diseases, caused for example by Rhizoctonia solani;
Sarocladium diseases caused for example by Sarocladium oryzae;
Sclerotium diseases caused for example by Sclerotium oryzae;
Tapesia diseases, caused for example by Tapesia acuformis;
Thielaviopsis diseases, caused for example by Thielaviopsis basicola;
   Ear and panicle diseases such as:
Alternaria diseases, caused for example by Alternaria spp.;
Aspergillus diseases, caused for example by Aspergillus flavus;
Cladosporium diseases, caused for example by Cladosporium spp.;
Claviceps diseases, caused for example by Claviceps purpurea;
Fusarium diseases, caused for example by Fusarium culmorum;
Gibberella diseases, caused for example by Gibberella zeae;
Monographella diseases, caused for example by Monographella nivalis;
   Smut and bunt diseases such as:
Sphacelotheca diseases, caused for example by Sphacelotheca reiliana;
Tilletia diseases, caused for example by Tilletia caries;
Urocystis diseases, caused for example by Urocystis occulta;
Ustilago diseases, caused for example by Ustilago nuda;
   Fruit rot and mould diseases such as:
Aspergillus diseases, caused for example by Aspergillus flavus;
Botrytis diseases, caused for example by Botrytis cinerea;
Penicillium diseases, caused for example by Penicillium expansum;
Rhizopus diseases caused by example by Rhizopus stolonifer
Sclerotinia diseases, caused for example by Sclerotinia sclerotiorum;
Verticilium diseases, caused for example by Verticilium alboatrum;
   Seed and soilborne decay, mould, wilt, rot and damping-off diseases:
Alternaria diseases, caused for example by Alternaria brassicicola
Aphanomyces diseases, caused for example by Aphanomyces euteiches
Ascochyta diseases, caused for example by Ascochyta lentis
Aspergillus diseases, caused for example by Aspergillus flavus
Cladosporium diseases, caused for example by Cladosporium herbarum
Cochliobolus diseases, caused for example by Cochliobolus sativus (Conidiaform: Drechslera, Bipolaris Syn: Helminthosporium);
Colletotrichum diseases, caused for example by Colletotrichum coccodes;
Fusarium diseases, caused for example by Fusarium culmorum;
Gibberella diseases, caused for example by Gibberella zeae;
Macrophomina diseases, caused for example by Macrophomina phaseolina
Monographella diseases, caused for example by Monographella nivalis;
Penicillium diseases, caused for example by Penicillium expansum
Phoma diseases, caused for example by Phoma lingam
Phomopsis diseases, caused for example by Phomopsis sojae;
Phytophthora diseases, caused for example by Phytophthora cactorum;
Pyrenophora diseases, caused for example by Pyrenophora graminea
Pyricularia diseases, caused for example by Pyricularia oryzae;
Pythium diseases, caused for example by Pythium ultimum;
Rhizoctonia diseases, caused for example by Rhizoctonia solani;
Rhizopus diseases, caused for example by Rhizopus oryzae
Sclerotium diseases, caused for example by Sclerotium rolfsii;
Septoria diseases, caused for example by Septoria nodorum;
Typhula diseases, caused for example by Typhula incarnata;
Verticillium diseases, caused for example by Verticillium dahliae;
   Canker, broom and dieback diseases such as:
Nectria diseases, caused for example by Nectria galligena;
   Blight diseases such as:
Monilinia diseases, caused for example by Monilinia laxa;
   Leaf blister or leaf curl diseases such as:
Exobasidium diseases caused for example by Exobasidium vexans
Taphrina diseases, caused for example by Taphrina deformans;
   Decline diseases of wooden plants such as:
Esca diseases, caused for example by Phaemoniella clamydospora;
Eutypa dyeback, caused for example by Eutypa lata;
Ganoderma diseases caused for example by Ganoderma boninense;
Rigidoporus diseases caused for example by Rigidoporus lignosus
   Diseases of Flowers and Seeds such as
Botrytis diseases caused for example by Botrytis cinerea;
   Diseases of Tubers such as
Rhizoctonia diseases caused for example by Rhizoctonia solani;
Helminthosporium diseases caused for example by Helminthosporium solani;
   Club root diseases such as
Plasmodiophora diseases, cause for example by Plamodiophora brassicae.
   Diseases caused by Bacterial Organisms such as
Xanthomonas species for example Xanthomonas campestris pv. oryzae;
Pseudomonas species for example Pseudomonas syringae pv. lachrymans;
Erwinia species for example Erwinia amylovora.

### LEGEND OF THE FIGURES

FIG. 1: Measurement of Phytophthora infestans growth inhibition in the presence of dsRNA targeting the saccharopine dehydrogenase messenger RNA.

FIG. 2: Analysis of saccharopine dehydrogenase mRNA level by qRT PCR.

### SEQUENCE LISTING

SEQ ID No 1: Saccharopine dehydrogenase (LYS1) from Aspergillus clavatus.

SEQ ID No 2: Protein encoded by the above nucleic acid sequence.

SEQ ID No 3: Saccharopine dehydrogenase (LYS1) from Aspergillus fumigatus.

SEQ ID No 4: Protein encoded by the above nucleic acid sequence.

SEQ ID No 5: Saccharopine dehydrogenase (LYS1) from Botrytis cinerea.

SEQ ID No 6: Protein encoded by the above nucleic acid sequence.

SEQ ID No 7: Saccharopine dehydrogenase (LYS1) from Fusarium graminearum.

SEQ ID No 8: Protein encoded by the above nucleic acid sequence.

SEQ ID No 9: Saccharopine dehydrogenase (LYS1) from Fusarium oxysporum.

SEQ ID No 10: Protein encoded by the above nucleic acid sequence.

SEQ ID No 11: Saccharopine dehydrogenase (LYS1) from Fusarium verticilloides.

SEQ ID No 12: Protein encoded by the above nucleic acid sequence.

SEQ ID No 13: Saccharopine dehydrogenase (LYS1) from Fusarium verticilloides.

SEQ ID No 14: Protein encoded by the above nucleic acid sequence.

SEQ ID No 15: Saccharopine dehydrogenase (LYS1) from Mycosphaerella fijiensis.

SEQ ID No 16: Polypeptide encoded by the above nucleic acid sequence.

SEQ ID No 17: Saccharopine dehydrogenase (LYS1) from Magnaporthe grisea.

SEQ ID No 18: Protein encoded by the above nucleic acid sequence.

SEQ ID No 19: Saccharopine dehydrogenase (LYS1) from Monoliophthora perniciosa.

SEQ ID No 20: Protein encoded by the above nucleic acid sequence.

SEQ ID No 21: Saccharopine dehydrogenase (LYS1) from Puccinia graminis.

SEQ ID No 22: Protein encoded by the above nucleic acid sequence.

SEQ ID No 23: Saccharopine dehydrogenase (LYS1) from Phytophthora infestans.

SEQ ID No 24: Protein encoded by the above nucleic acid sequence.

SEQ ID No 25: Saccharopine dehydrogenase (LYS1) (from Phytophthora ramorum.

SEQ ID No 26: Protein encoded by the above nucleic acid sequence.

SEQ ID No 27: Saccharopine dehydrogenase (LYS1) from Phytophthora sojae.

SEQ ID No 28: Protein encoded by the above nucleic acid sequence.

SEQ ID No 29: Saccharopine dehydrogenase (LYS1) from Pyrenophora tritici-repentis.

SEQ ID No 30: Protein encoded by the above nucleic acid sequence.

SEQ ID No 31: Saccharopine dehydrogenase (LYS1) from Sclerotinia sclerotiorum.

SEQ ID No 32: Protein encoded by the above nucleic acid sequence.

SEQ ID No 33: Saccharopine dehydrogenase (LYS1) from Trichoderma reesei.

SEQ ID No 34: Protein encoded by the above nucleic acid sequence.

SEQ ID No 35: Saccharopine dehydrogenase (LYS1) from Ustilago maydis.

SEQ ID No 36: Protein encoded by the above nucleic acid sequence.

SEQ ID No 37: Saccharopine dehydrogenase (LYS1) from Verticillium albo-atrum.

SEQ ID No 38: Protein encoded by the above nucleic acid sequence.

SEQ ID No 39: Saccharopine dehydrogenase (LYS1) from Mycosphaerella graminicola.

SEQ ID No 40: Protein encoded by the above nucleic acid sequence.

SEQ ID No 41: Saccharopine dehydrogenase (LYS1) from Fusarium moniliform.

SEQ ID No 42: Protein encoded by the above nucleic acid sequence.

SEQ ID No 43: Saccharopine dehydrogenase (LYS1) from Claviceps purpurea.

SEQ ID No 44:Protein encoded by the above nucleic acid sequence.

SEQ ID No 45: Primer SACdh_Pi_T7_F

SEQ ID No 46: Primer SACdh_Pi_T7_R

SEQ ID No 47: Primer Actin forward

SEQ ID No 48: Primer Actin reverse

SEQ ID No 49: Primer β-Tub forward

SEQ ID No 50: Primer β-Tub reverse

SEQ ID No 51: Primer SACdh forward

SEQ ID No 52: Primer SACdh reverse

SEQ ID No 53: Primer pBINB33-1

SEQ ID No 54: Primer pBINB33-2

SEQ ID No 55: Primer SacdhPI R

SEQ ID No 56: Primer SacdhPI F

SEQ ID No 57: Primer LYS1 Pot 117-F

SEQ ID No 58: Primer LYS1 Pot 117-R

The various aspects of the invention will be understood more fully by means of the experimental examples below.

All the methods or operations described below are given by way of example and correspond to a choice, made among the various methods available for achieving the same result. This choice has no effect on the quality of the result, and, consequently, any appropriate method can be used by those skilled in the art to achieve the same result. In particular, and unless otherwise specified in the examples, all the recombinant DNA techniques employed are carried out according to the standard protocols described in Sambrook and Russel (2001, Molecular cloning: A laboratory manual, Third edition, Cold Spring Harbor Laboratory Press, NY) in Ausubel et al. (1994, Current Protocols in Molecular Biology, Current protocols, USA, Volumes 1 and 2), and in Brown (1998, Molecular Biology LabFax, Second edition, Academic Press, UK). Standard materials and methods for plant molecular biology are described in Croy R. D. D. (1993, Plant Molecular Biology LabFax, BIOS Scientific Publications Ltd (UK) and Blackwell Scientific Publications (UK)). Standard materials and methods for PCR (Polymerase Chain Reaction) are also described in Dieffenbach and Dveksler (1995, PCR Primer: A laboratory manual, Cold Spring Harbor Laboratory Press, NY) and in McPherson et al. (2000, PCR—Basics: From background to bench, First edition, Springer Verlag, Germany).

### REF BIBLIO

Bevan, 1984, Nucl Acids Res 12: 8711-8721
Bhattacharjee, J. K., 1985, Crit. Rev. Microbiol. 12, 131-151
Bhattacharjee, J. K., 1992, in Handbook of Evolution of Metabolic Function, Mortlock, R. P., ed., CRC Press, Boca Raton, Fla., pp. 47-80
Born T. L. and Blanchard J. S., 1999, Curr. Opin. Chem. Biol., 3:607-613
Broquist, H. P., 1971, Methods Enzymol. 17, 112-129
Chuang and Meyerowitz, 2000, PNAS, 97: 4985-4990
Ehmann et al., 1999, biochemistry, 38, 6171-6177
Escobar et al., 2001, Proc. Natl. Acad. Sci. USA., 98(23): 13437-13442
Fire et al. 1998, Nature 391: 806-811
Garrad, R. C. and Bhattacharjee, J. K., 1992, J. Bacteriol. 174, 7379-7384
Gielen et al, 1984, EMBO Journal 3, 835-846
Hamilton and Baulcome, 1999, Science 286: 950-952
Hamilton et al., 1998, Plant J, 15: 737-746
Hammond et al., 2000, Nature 404: 293-296
Hannon, 2002, Nature, 418 (6894): 244-51
Herrera-Estrella et al, 1983 Nature, 303, 209-213
Hofgen and Willmitzer, 1990, Plant Science 66, 221-230
Houmard et al., 2007, Plant Biotechnology Journal 5, 605-614
Montgomery et al., 1998, PNAS 95: 15502-15507
Pandolfini et al., 2003, Biotechnol., 25; 3(1): 7
Pietrzak et al, 1986 Nucleic Acids Res. 14, 5858
Thompson et al., Nucleic Acids Research 22 (1994), 4673-4680
Umbargar, H. E., 1978, Annu. Rev. Biochem. 47, 533-606
Randall, T. A., 2005, MPMI, 18, 229-243
Rocha-Sosa et al., 1989, EMBO J. 8, 23-29
Ruiz Ferrer and Voinnet, Annu. Rev. Plant Biol. 2009. 60:485-510
Sambrook et al., 2001, Molecular Cloning: A Laboratory Manual, third edition, ISBN978-087969577-4
Silué et al. Physiol. Mol. Plant Pathol., 53 239-251, 1998
Stärkel C., Ph. D. thesis “Host induced gene silencing—strategies for the improvement of resistance against Cercospora beticola in sugar beet (B. vulgaris L) and against Fusarium graminearum in wheat (T. aestivum L.) and maize (Z. mays L.)”, defended in June 2011, XP002668931, retrieved from internet (2012 Feb. 7) on http://ediss.subμni-hamburg.de/vol|teste/2011/5286/pdf/Dissertation.pdf
Tang et al., 2003 Gene Dev., 17(1): 49-63
Villalba et al. Fungal Genetics and Biology 45, 68-75, 2008
Vogel, H. J., 1965, in Handbook of Evolving Genes and Proteins, Bryson, V., ed., Academic Press, New York, pp. 25-40
Waterhouse et al., 1998, PNAS 95: 13959-13964,
Wesley et al. The Plant Journal (2001); 27(6), 581-590
Xiao et al., 2003, Plant Mol Biol., 52(5): 957-66
Xu et al., 2006, Cell Bio chemistry and Biophysics, vol 46, 43-64
Zamore et al., 2000, Cell, 101: 25-33,

### EXAMPLES

### Example 1: In Vitro Cultivation of Magnaporthe grisea

Assays were carried out using the Magnaporthe grisea wild-type strain P1.2 originated from the collection of the phytopathology laboratory of the CIRAD (Centre de coopération internationale en recherche agronomique pour le développement) in Montpellier. Conditions for culturing, the composition of the rice-agar medium, maintenance, and sporulation as well as protoplasts preparations are described by Silué et al. (1998).

### Example 2: Magnaporthe grisea Transfection with dsRNA Targeting Saccharopine Dehydrogenase and Measurement of Growth Inhibition

The Magnaporthe grisea saccharopine dehydrogenase (SACdh) gene sequence Lys-1 (MGG_01359.6: 1426 bp) was obtained from the Broad Institute (http://www.broadinstitute.org/). A region of about 325 bp was selected for dsRNA, comprising the nucleotides 301 through 626, was synthesized by the Geneart company and cloned into the plasmid.

For transfection, ds RNA of saccharopine dehydrogenase from Magnaporthe grisea was produced using the MEGAscript RNAi Kit (Ambion) according to the manufacturers' instructions. Different amounts (200 μg to 2 μg of ds RNA) were treated with transfection agent Lipofectamin RNAi max (Invitrogen) following manufactures' instructions. Lipofectamin-ds RNA complexes were added to 2.5×10⁶ Magnaporthe grisea protoplast in a microtiterplate with TB3 media (Villalba et al., 2008), and growth was monitored for 5-7 days at a OD of 600 nm using a Infinite M1000 (Tecan) microplate reader.

Growth of Magnaporthe grisea protoplasts treated with ds RNA of saccharopine dehydrogenase comparing to untreated control. was monitored at several time points and showed a significant difference in growth.

### Example 3: In Vitro Cultivation Phytophthora infestans and Zoospores Preparation

Phytophthora infestans strain PT78 was cultivated in vitro in 9 cm petri dishes on pea agar medium (125 g/l boiled and crushed peas, 20 g/l agar agar, carbenicillin 100 mg/l) at 21° C. in the dark. Every 15 days, new medium was inoculated with four 5 mm cubic plugs of mycelium.

To release the zoospores, 12 ml of ice cold water were put on a 10 days old culture and the culture was placed at 4° C. for 2 hours. The supernatant was then collected without disturbing the mycelium and filtered through a 100 μm sieve to remove hyphal fragments. The zoospores were placed on ice and counted with a haemocytometer.

### Example 4: Phytophthora infestans Transfection with dsRNA Targeting Saccharopine Dehydrogenase and Measurement of Growth Inhibition

The Phytophthora infestans saccharopine dehydrogenase gene sequence Lys-1 (PITG_03530: 3020 bp) was obtained from the P. infestans database of the Broad Institute. A region about 500 bp offering the best siRNA according to the BLOCKit RNAi designer software (Invitrogen) and comprising the nucleotides 2251 through 2750, was synthesized by the Geneart company and cloned into the plasmid 0920357_SacDH_Pi_pMA.

Synthesis of dsRNA was carried out using the Megascript RNAi kit (Ambion) following the manufacturer's protocol and using as a template a PCR product amplified from the plasmid 0920357_SacDH_Pi_pMA. The forward primer used was SACdh_Pi_T7_F: 5′ TAATACGACTCACTATAGGGTTGCAGGAGAGCGCAGAAAGC (SEQ: ID NO: 45) and the reverse primer was SACdh_Pi_T7_R: TAATACGACTCACTATAGGGTCAGTTGGAGTCCGCGTGGTGT (SEQ. ID NO: 46).

dsRNA were then precipitated with 100% ethanol and sodium acetate 3M, pH5.2, washed 2 times with 70% ethanol and the pellets were resuspended in RNase free water.

The transfection mixes were prepared in a 48 well plate by adding sequentially V8 medium (5% of V8 juice (Campbell Foods Belgium), pH5), the appropriate amount of dsRNA and 10 μl of lipofectamine RNAi max (Invitrogen) in a final volume of 200 μl. The transfection mixture was incubated during 15 min at room temperature.

Zoospores were diluted in V8 medium to a concentration of 5×10⁴ zoospores/ml. Then, 800 μL of the zoospores solution were added in each well of the plate. The final concentration of zoospores was 4×10⁵ zoospores/ml.

Three controls were added on each plate: V8 medium, V8 medium+zoospores, V8 medium+zoospores+lipofectamine. The plates were incubated at 21° C., in the dark.

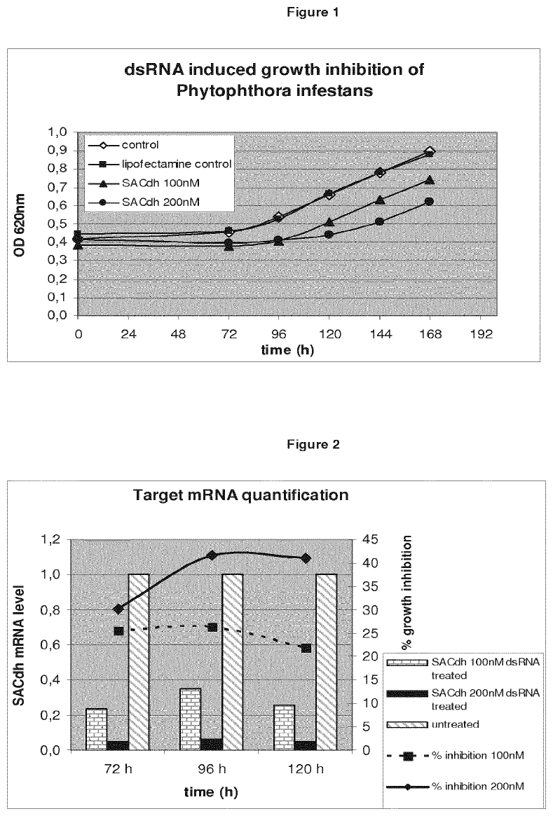

The growth of the fungus was followed by measuring the absorbance at 620 nm in a plate reader (Infinite 1000, Tecan) over 8 days The percentage of growth inhibition was calculated using the following formula: 100−(OD_{dsRNA}×100/OD_{control lipofectamine}). The growth of the fungi was reduced in the presence of dsRNA directed against saccharopine dehydrogenase in a concentration dependant manner (100 nM and 200 nM respectively) as shown in FIG. 1

### Example 5: Quantitative PCR Analysis of P. infestans Saccharopine Dehydrogenase Messenger RNA

To yield sufficient RNA for cDNA synthesis and real-time RT-PCR, several wells of the 48 wells plate were pooled for one concentration of dsRNA tested: 10 wells for 72 h time point, 6 wells for 96 h time point, 3 wells for 120 h time point.

After 72 h, 96 h and 120 h of treatment with the dsRNA, the mycelia were collected. The samples were centrifuged to remove the medium. The samples were frozen in liquid nitrogen and then lyophilized overnight.

Before RNA extraction, the mycelium was grinded. Total RNA was extracted using the RNeasy Plant mini kit (Qiagen) following the manufacturer's protocol. DNA contamination of the RNA samples was removed by DNase digestion (DNA free, Ambion). Integrity of the RNA was tested on the 2100 Bioanalyzer (RNA 6000 nano kit, Agilent) following the protocol supplied by the manufacturer. The cDNA were synthesized from 2 μg of total RNA by oligo dT priming using the kit Thermoscript RT-PCR system (Invitrogen) following the manufacturer's protocol. The cDNA were precipitated with 100% EtOH and sodium acetate 3M, pH5.2, washed 2 times with 70% EtOH and the pellets were resuspended in 10 μL of RNase free water. The cDNA were diluted a hundred fold for the qPCR test. Primer pairs were designed for each gene sequence by using the Primer Express 3 software (Applied Biosystems). Real time RT-PCR was performed on a 7900 Real Time PCR system (Applied Biosystems) with Power SYBR green PCR master mix (Applied Biosystems) following the manufacturer's protocol. Q-PCR was performed as follows: 95° C. for 10 min, 45 cycles at 95° C. for 15 s and 60° C. 1 min, followed by a dissociation stage at 95° C. for 15 s, 60° C. for 1 min and 95° C. for 15 s.

The actin and β3-tubuline genes were used as endogenous controls. The relative expression of genes was calculated with the 2ΔΔCt method. The FIG. 2 shows a significant reduction of the level of the saccharopine dehydrogenase messenger RNA and this reduction correlates with growth inhibition.

TABLE 1
Sequence of qPCR primers :

| Primer name | Forward primer sequence | Reverse primer sequence |
|---|---|---|
| actin | CGACTCTGGTGACGGTGTGT | GCGTGAGGAAGAGCGTAACC |
| | (SEQ ID NO: 47) | (SEQ ID NO: 48) |
| β-Tub | CCGCCCAGACAATTTCGT | CCTTGGCCCAGTTGTTACCA |
| | (SEQ ID NO: 49) | (SEQ ID NO: 50) |
| SACdh | TGGGTGGTTTCCAAGGTCTTC | AAAGGCACCAAGCCACTGAA |
| | (SEQ ID NO 51) | (SEQ ID NO: 52) |

### Example 6: Construction of Transformation Vectors Containing the Phytophthora infestans Saccharopine Dehydrogenase Gene

### a) Preparation of the Plant Expression Vector IR 47-71

The plasmid pBinAR is a derivative of the binary vector plasmid pBin19 (Bevan, 1984) which was constructed as follows: A fragment of a length of 529 bp which comprised the nucleotides 6909-7437 of the 35S promoter of the cauliflower mosaic virus was isolated as EcoR \IKpn I fragment from the plasmid pDH51 (Pietrzak et al, 1986) and ligated between the EcoR I and Kpn I restriction sites of the polylinker of pUC18. In this manner, the plasmid pUC18-35S was formed. Using the restriction endonucleases Hind III and Pvu II, a fragment of a length of 192 bb which included the

polyadenylation signal (3′ terminus) of the Octopin Synthase gene (gene 3) of the T-DNA of the Ti plasmid pTiACHσ (Gielen et al, 1984) (nucleotides 11 749-11 939) was isolated from the plasmid pAGV40 (Herrera-Estrella et al, 1983). Following addition of Sph I linkers to the Pvu II restriction site, the fragment was ligated between the Sph I and Hind III restriction sites of pUC18-35S. This gave the plasmid pA7. Here, the entire polylinker comprising the 35S promoter and Ocs terminator was removed using EcoR I and Hind III and ligated into the appropriately cleaved vector pBin19. This gave the plant expression vector pBinAR (Hofgen and VVilimitzer, 1990).

The promoter of the patatin gene B33 from Solanum tuberosum (Rocha-Sosa et al., 1989) was, as Dra I fragment (nucleotides-1512-+14), ligated into the Ssf I-cleaved vector pUC19 whose ends had been blunted using T4-DNA polymerase. This gave the plasmid pUC19-B33. From this plasmid, the B33 promoter was removed using EcoR I and Sma I and ligated into the appropriately restricted vector pBinAR. This gave the plant expression vector pBinB33. To facilitate further cloning steps, the MCS (Multiple Cloning Site) was extended. To this end, two complementary oligonucleotides were synthesized, heated at 95° C. for 5 minutes, slowly cooled to room temperature to allow good fixation (annealing) and cloned into the Sal I and Kpn I restriction sites of pBinB33. The oligonucleotides used for this purpose had the following sequence:

pB1NB33-1: 5′-TCG ACA GGC CTG GAT CCT TAA TTA AAC TAG TCT CGA GGA GCT CGG TAC-3′ (SEQ ID NO: 53) pB1 NB33-2: 5′-CGA GCT CCT CGA GAC TAG TTT AAT TAA GGA TCC AGG CCT G-3′
(SEQ ID NO: 54) The plasmid obtained was named IR 47-71.
b) Preparation of the Plant Expression Vectors pEPA248 and pEPA262 Comprising a Nucleic Acid Sequences for the Phytophthora infestans Saccharopine Dehydrogenase Gene.

The saccharopine dehydrogenase sequence (PITG_03530: 3020 bp) was obtained from the P. infestans ORF Prot V1 database. A region about 500 bp offering the best siRNA according to the BLOCKit RNAi designer software (Invitrogen), was synthesized by the Geneart company.

A 300 bp fragment was amplified by PCR from this sequence DNA with the primers SacdhPI R (5′-agaggtaccaagcttgcgtagctgg-3′) and SacdhPI F (5′-tatctcgagtctagacaacgccattggttac-3′). The amplified fragment was cloned into pCRII-Topo (Invitrogen) to obtain the plasmid pEPA250. According to Wesley et al. (2001), the sequence of interest was cloned in pHannibal vector to give plasmid pEPA241. Then the dsRNA expression cassette was sub-cloned into different binary (plant expression) vectors pART27 (Gleave A P, PMB 20, (1992), 1203-1207) and IR 47 to produce the plant expression vectors pEPA248 and pEPA262, respectively.

Vector pEPA248 and pEPA262 were introduced into respectively GV3101 and C58C1RIF (pGV2260) agrobacteria cells by electroporation (Rocha-Sosa et al. (1989)), in order to further transform potato plants.

### Example 7: Construction of Transformation Vectors Targeting the Sclerotinia scharotiorum Saccharopine Dehydrogenase Gene LysI

The 351 bp of a region of the S. sclerotiorum Lysi coding sequence (saccharopine dehydrogenase SS1G_06166.1) was synthesized by the Geneart company (pEPA293), and flanked by internal (XbaI, HindIII) and external (XhoI, KpnI) restriction sites designed to perform a two-step cloning into the pHannibal vector (Wesley et al., 2001). The intermediate plasmid harbored two inverted copies of the LysI gene fragment spaced by the pHannibal PdK intron and regulated by the cauliflower mosaic virus (CaMV) 35S promoter and the OCS terminator.

The entire DNA cassette was then excised with NotI and inserted into the pART27 binary vector (Gleave, 1992), giving the final plasmid pEPA307 with a plant selection cassette based on kanamycin resistance (nptII gene regulated by the Nos promoter and terminator).

The same NotI cassette was also inserted in a binary vector (pFC031) with a plant selection marker based on an HPPD inhibitors resistance, to be used in Soybean transformation. The final plasmid can then transform plants with a T-DNA comprising in between the Right and Left borders, our cassette of interest and an HPPD gene regulated by a CsVMV promoter, a chloroplast transit peptide sequence and a 3′Nos terminator.

### Example 8: Construction of Transformation Vectors Targeting the Phakopsora pachirizi Saccharopine Dehydrogenase Gene Lys1

The 364 bp of a region of a Phakopsora pachirizi LysI E.S.T. (saccharopine dehydrogenase PHAPC_EH247326.1) was synthesized by the Geneart company (pCED42), and flanked by internal (XbaI, HindIII) and external (XhoI, KpnI), restriction sites designed to perform a two-step cloning into the pHannibal vector (Wesley et al., 2001). The intermediate plasmid harbored two inverted copies of the Lys1 gene fragment spaced by the pHannibal PdK intron and regulated by the cauliflower mosaic virus (CaMV) 35S promoter and the OCS terminator.

The entire DNA cassette was then excised with NotI and inserted into the pART27 binary vector (Gleave, 1992), giving the final plasmid pCED45 with a plant selection cassette based on kanamycin resistance (nptII gene regulated by the Nos promoter and terminator).

The same NotI cassette was also inserted in a binary vector (pFCO31) with a plant selection marker based on an HPPD inhibitors resistance, to be used in Soybean transformation. The final plasmid (pCED87) can then transform plants with a T-DNA comprising inbetween the Right and Left borders, our cassette of interest and an HPPD gene regulated by a CsVMV promoter, a chloroplast transit peptide sequence and a 3′Nos terminator.

### Example 9: Transformation of Potato Plants with Plant Expression Vectors Comprising Nucleic Acid Molecules Coding for Hairpin Saccharopine Dehydrogenase Construct pEPA262

Potato plants were transformed via Agrobacterium using the plant expression vector pEPA262, which comprises a coding nucleic acid sequence for saccharopine dehydrogenase under the control of the promoter of the patatin gene B33 from Solanum tuberosum as described by Rocha-Sosa et al. (1989). The transgenic potato plants transformed with the plasmid pEPA262, were named “537 ES”. Molecular analysis of the events of “537 ES” was performed using standard PCR methods (Sambrook et al.) to detect the presence of the nucleic acid sequence for saccharopine dehydrogenase using the following primers SacDH PI F: 5′-TATCTCGAGTCTAGACAACGCCATTGGTTAC-3′ (SEQ ID NO: 56) and SacDH PI R: 5′-AGAGGTACCAAGCTTGCGTAGCTGG-3′. (SEQ ID NO: 55) Further selection was accomplished either by Northern blotting or by expression analysis of the nucleic acid sequence for saccharopine dehydrogenase via RT-Q PCR leading to a selection of different events. The oligonucleotides used for this purpose had the following sequence: LYS1_Pot 117-F: 5′-TCA ATA GAA GCG AAC GCG TAA A-3′ (SEQ ID NO: 57) and LYS1_Pot 117-R: 5′-GTT CGG GAT CTG CTC GAT GT-3′ (SEQ. ID NO: 58).

### Example 10: Agrobacterium-Mediated Transformation of Arabidopsis thaliana

The pART27 derived plasmids was introduced into Agrobacterium tumefaciens strain LBA4404 (Invitrogen Electromax) by electroporation. The obtained bacterial strains were then used for the floral dip infiltration of the A. thaliana Col-0 or Wassileskija plants as described by Clough & Bent (PlantJ 1998).

### Example 11: Agrobacterium-Mediated Transformation of Glycine max

The pFCO31 derived plasmids were introduced into Agrobacterium tumefaciens strain LBA4404 (Invitrogen Electromax) by electroporation. The obtained bacterial strains were then used for Soybean transformation as described below.

Soybean seeds are sterilized for 24 h with Chlorine gas (Cl2). Seeds are then placed in Petri dishes and soaked in sterile deionized water for 20 hours prior to inoculation, in the dark, at room temperature. An overnight culture grown at 28° C. and 200 rpm agitation of Agrobacterium tumefaciens in 200 ml of YEP (5 g/L Yeast extract, 10 g/L Peptone, 5 g/L NaCl2. pH to 7.0) containing the appropriate antibiotic is centrifugated at 4000 rpm, 4° C., 15 min. The pellet is resuspended in 40 to 50 mL of infection medium to a final OD600 nm between 0.6 and 1 and stored on ice. Soaked seeds are dissected, under sterile conditions, using a #15 scalpel blade to separate the cotyledons and remove the primary leaves attached to them. Each cotyledon is kept as explant for inoculation. About 100 explants are prepared and subsequently inoculated together, for 30 minutes in the Agrobacterium inoculum, with occasional agitation. Cocultivation is performed in classical Petri dishes containing 4 papers filter (Whatman® grade 1) and 4 mL of Cocultivation medium ( 1/10×B5 major salts, 1/10×B5 minor salts, 2.8 mg/L Ferrous, 3.8 mg/L NaEDTA, 30 g/L Sucrose, 3.9 g/L MES (pH 5.4). Filter sterilized 1×B5 vitamins, GA3 (0.25 mg/L), BAP (1.67 mg/L), Cysteine (400 mg/L), Dithiothrietol (154.2 mg/L), and 200 μM acetosyringone). Explants are placed on co-cultivation plates (9 per plate), adaxial (flat) side down and sealed with a single vertical string of tape (Leucopore®) and further incubated for 5 days, at 24′C, in a 18:6 photoperiod. At the end of cocultivation, the explants are placed (6 per plate) on the Shoot Initiation Medium (1×B5 major salts, 1×B5 minor salts, 28 mg/L Ferrous, 38 mg/L NaEDTA, 30 g/L Sucrose, 0.56 g/L MES, and 8 g/L agar (pH 5.6). Filter sterilized 1×B5 vitamins, BAP (1.67 mg/L), Timentin (50 mg/L), Cefotaxime (50 mg/L), Vancomycin (50 mg/L) and Tembotrione (0.1 mg/L)), inclined at 45°, with the cotyledonary node area imbedded in the medium and upwards. The Shoot Initiation step lasts 1 month (24° C. 16/8 photoperiod). After one more month, explants with green shoots are transferred on Shoot Elongation Medium (1×MS/B5 medium amended with 1 mg/l zeatin riboside (ZR), 0.1 mg/l IAA, 0.5 mg/l GA3, 3% sucrose, 100 mg/l pyroglutamic acid, 50 mg/l asparagine, 0.56 g/L MES, pH 5.6, solidified with 0.8% agar, ticarcillin (50 mg/l), cefotaxime (50 mg/l) and vancomycin (50 mg/L)), with fresh transfer every 2 weeks. Plantlets that are more than 2 cm high are transferred on Rooting medium. Plantlets are cut and placed on rooting medium (½ MS major salts, minor salts and vitamins B5, 15 g/L Sucrose, 1 mg/L IBA 8 g/L Noble agar, pH 5.7). in an 180 mL vertical plastic container.

Once the roots are well formed and the apex is strong, plants are placed into soil in the greenhouse and covered with a green plastic box for acclimatization for 5 days on a 36° C. heating bed. After 10 days of acclimatization, the plants are transferred into big pots, without heating bed.

### Example 12: Asian Soybean Rust (Phakopsora pachyrhizi) Assay

Soybean plants expressing dsRNA directed against Phakopsora pachirizi Lys1 were grown in the greenhouse in 7.5 cm pots (28.5° C., 50% humidity, 1 4 h light). In an incubator, plants were sprayed with a conidia suspension (50 ml at 10-15×10⁴ spores/ml obtained from artificially infected soybean plants serving as a source of inoculum, for one tray of dimensions 55×34×5 cm containing 15 pots). Suspension includes Tween20 at 0.033%. To ensure even inoculation multidirectional spraying is necessary. Plants are then incubated for 4 days at ca. 25° C. (daytime) and ca. 20° C. (night) with very high humidity (90% to saturation). After this period plants are transferred back to normal growing conditions. Asian soybean rust development is evaluated at regular intervals to follow kinetics of disease development and severity of symptoms. All experiments with Asian soybean rust are performed in L2 safety level culture chambers or incubators according to HCB requirements.

### Example 13: Sclerotinia sclerotiorum Assay

Development of the Wild-type S. sclerotiorum isolate 1980 as well as the pac 1 mutant (Rollins, 2003) fungus was studied on a whole plant assay. S. sclerotiorum was stored at 4° C. on potato dextrose agar (PDA, potato 200 g/I, glucose 20 g/I, agar 18 g/l). The fungus was cultured in a Petri dish containing PDA by placing a mycelial plug in the centre and was maintained under static conditions at 21° C. for 4 days. 4 weeks old Arabidopsis wild-type and transgenic plants were inoculated with 12-mm diameter agar-mycelium plugs excised from the actively growing margin of the fungal colony in the centre of the plant. Inoculated plants were kept in a growth chamber at 21° C. with 100% relative humidity under a 12-h light photoperiod with a light intensity of 34 mmol m-2 s-1 using fluorescent white lights and were monitored every 12 h to observe fungal development. Disease symptoms were monitored by number of infected leaves as well as lengths and widths of lesions.

1. A dsRNA molecule comprising i) a first strand comprising a sequence substantially identical to at least 20 contiguous nucleotides of a fungus or oomycete saccharopine dehydrogenase gene and ii) a second strand comprising a sequence substantially complementary to the first strand.

2. The dsRNA molecule according to claim 1, wherein the fungus or oomycete gene is:
a) a polynucleotide comprising a sequence as set forth in SEQ ID NO: 1, 3, 5, 7, 9, 11, 13, 15, 17, 19, 21, 23, 25, 27, 29, 31, 33, 35, 37, 39, 41, or 43;
b) a polynucleotide encoding a polypeptide having a sequence as set forth in SEQ ID NO: 2, 4, 6, 8, 10, 12, 14, 16, 18, 20, 22, 24, 26, 28, 30, 32, 34, 36, 38, 40, 42, or 44;
c) a polynucleotide having at least 70% sequence identity to a polynucleotide having a sequence as set forth in SEQ ID NO: 1, 3, 5, 7, 9, 11, 13, 15, 17, 19, 21, 23, 25, 27, 29, 31, 33, 35, 37, 39, 41, or 43; or
d) a polynucleotide encoding a polypeptide having at least 70% sequence identity to a polypeptide having a sequence as set forth in SEQ ID NO: 2, 4, 6, 8, 10, 12, 14, 16, 18, 20, 22, 24, 26, 28, 30, 32, 34, 36, 38, 40, 42, or 44.

3. A composition comprising at least a dsRNA molecule according to claim 1.

4. The composition according to claim 3, further comprising an agriculturally acceptable support, carrier, filler and/or surfactant.

5. The composition according to claim 3, further comprising a phytopharmaceutical or plant growth promoting compound.

6. A micro-organism producing a dsRNA molecule according to claim 1.

7. A genetic construct comprising at least one DNA sequence as well as heterologous regulatory element(s) in the 5′ and optionally in the 3′ positions, wherein the DNA sequence(s) is able to form a dsRNA molecule according to claim 1.

8. A cloning and/or expression vector, comprising at least one genetic construct according to claim 7.

9. A transgenic plant cell capable of expressing at least a dsRNA molecule according to claim 1.

10. A transgenic plant, seed or part thereof, comprising a transgenic plant cell according to claim 9.

11. The transgenic plant cell according to claim 9, or transgenic plant, seed or part thereof, comprising said transgenic plant cell, wherein said plant is a soybean, oilseed, rice or potato plant.

12. A method of making a transgenic plant cell capable of expressing a dsRNA that inhibits a fungus or oomycete saccharopine dehydrogenase gene, comprising transforming a plant cell with a genetic construct according to claim 7.

13. A method of controlling a plant pathogen, comprising providing to said pathogen a dsRNA molecule according to claim 1, or a composition comprising said dsRNA molecule.

14. A method for controlling a plant pathogen, comprising applying an effective quantity of a dsRNA molecule according to claim 1 or a composition comprising said dsRNA molecule to soil where plants grow or are capable of growing, to the leaves and/or the fruit of plants or to the seeds of plants.

15. A method of controlling a plant pathogen, comprising providing a transformed plant cell according to claim 9 to a host plant of said plant pathogen.

16. A method for inhibiting the expression of a plant pathogen gene, comprising:
i) transforming plant cells with a genetic construct according to claim 7,
ii) placing the transformed cells under conditions that allow the transcription of said construct, and
iii) contacting the plant pathogen with the cells.

17. The method according to claim 13, wherein said plant pathogen is Magnaporthe grisea, Phytophthora infestans Sclerotinia sclerotinium or Phakopsora pachyrhizi.

18. The method according to claim 12, wherein said plant cell is soybean, oilseed, rice or potato.

19. The method of claim 13, wherein the plant pathogen is a fungus or oomycete.

20. The method of claim 14, wherein the plant pathogen is a fungus or oomycete.
